(19) United States
(12) Patent Application Publication
(10) Pub. No.: US 2025/0041006 A1
(43) Pub. Date: Feb. 6, 2025

(54) ROBOT DEVICE AND ROBOT SYSTEM

(71) Applicant: SONY GROUP CORPORATION, TOKYO (JP)

(72) Inventor: HIROYUKI SUZUKI, TOKYO (JP)

(21) Appl. No.: 18/716,391

(22) PCT Filed: Nov. 4, 2022
(86) PCT No.: PCT/JP2022/041127

§ 371 Date: Jun. 4, 2024

(30) Foreign Application Priority Data
Dec. 13, 2021 (JP) 2021-201493
May 10, 2022 (JP) 2022-077437

(51) Int. Cl. A61B 34/37; A61B 34/30; A61F 9/007; B25J 9/10

(52) U.S. Cl. CPC A61B 34/37; B25J 9/10; A61B 2034/302; A61F 9/007

(57) ABSTRACT

A robot device (2) includes a first robot (R1) including a base portion (4) and a distal end portion (5) and a second robot (R2) supported by the distal end portion (5) of the first robot (R1), the second robot (R2) supporting a surgical tool (T) to be inserted into the body of a patient, in which the first robot (R1) is operated by a user (U) by directly applying a force to the first robot (R1).

### FIELD

The present disclosure relates to a robot device and a robot system.

### BACKGROUND

For example, Patent Literature 1 discloses a device connected to a micropositioning arm and connected to a surgical tool.

### CITATION LIST

### Patent Literature

Patent Literature 1: JP 2021-106894 A

### SUMMARY

### Technical Problem

For example, it is necessary to align an insertion point into a human body of a surgical tool with a remote center of motion (RCM). There is room for study not only on the remote center of motion but also on alignment technology of surgical tools.

One aspect of the present disclosure provides a robot device and a robot arm system capable of facilitating alignment of a surgical tool.

### Solution to Problem

A robot device according to one aspect of the present disclosure includes: a first robot including a base portion and a distal end portion; and a second robot supported by the distal end portion of the first robot, the second robot supporting a surgical tool to be inserted into a body of a patient, wherein the first robot is operated by a user by directly applying a force to the first robot.

A robot system according to one aspect of the present disclosure includes: a robot device; and a support device, wherein the robot device includes: a first robot including a base portion and a distal end portion; and a second robot supported by the distal end portion of the first robot, the second robot supporting a surgical tool to be inserted into a body of a patient, the first robot is operated by a user by directly applying a force to the first robot, and the support device supports operation of the first robot by the user for aligning an insertion point of the surgical tool and a remote center of motion of the surgical tool.

### BRIEF DESCRIPTION OF DRAWINGS

FIG. 1 is a diagram illustrating a schematic configuration example of a robot system 1 according to an embodiment.

FIG. 2 is a diagram illustrating an example of arrangement of a robot device 2 with respect to a patient.

FIG. 3 is a diagram illustrating an example of a schematic structure of a robot R1.

FIG. 4 is a diagram schematically illustrating an example of transmission of a braking force by a transmission 7.

FIG. 5 is a diagram illustrating an example of detection of a positional deviation between an insertion point I of a surgical tool T and a remote center of motion RCM.

FIG. 6 is a diagram illustrating an example of detection of a positional deviation between the insertion point I of the surgical tool T and the remote center of motion RCM.

FIG. 7 is a diagram illustrating an example of marking of the surgical tool T.

FIG. 8 is a flowchart illustrating an example of alignment between the insertion point I of the surgical tool T and the remote center of motion RCM using the marker M.

FIG. 9 is a diagram schematically illustrating an example of the schematic structure of the support arm device 20 according to the embodiment.

FIG. 10 is a diagram schematically illustrating an example of the schematic structure of the support arm device 20 according to the embodiment.

FIG. 11 is a diagram schematically illustrating an example of a schematic structure of a connection mechanism 22A according to a modification.

FIG. 12 is a diagram schematically illustrating an example of a schematic structure of a connection mechanism 22B according to a modification.

FIG. 13 is a diagram schematically illustrating an example of a schematic structure of a connection mechanism 22C according to a modification.

FIG. 14 is a diagram illustrating an example of a schematic structure of a parallel link mechanism 21 and a connection mechanism 22 according to a modification.

FIG. 15 is a diagram illustrating the example of the schematic structure of the parallel link mechanism 21 and the connection mechanism 22 according to the modification.

FIG. 16 is a diagram illustrating an example of assembly of the parallel link mechanism 21 and the connection mechanism 22.

FIG. 17 is a diagram illustrating an example of the schematic structure of the parallel link mechanism 21 and the connection mechanism 22 that are assembled.

FIG. 18 is a diagram illustrating an example of schematic structures of a robot R1 and a robot R2.

FIG. 19 is a diagram illustrating an example of a schematic structure of the robot device 2.

FIG. 20 is a diagram illustrating an example of the schematic structure of the robot device 2.

FIG. 21 is a schematic diagram illustrating a schematic structure example of a retraction mechanism.

FIG. 22 is a diagram for explaining a force applied to each unit in the structure illustrated in FIG. 21.

FIG. 23 is a diagram for explaining the operation principle of a retraction mechanism 101.

FIG. 24 is a diagram for explaining the operation principle of the retraction mechanism 101.

FIG. 25 is a diagram for explaining the operation principle of the retraction mechanism 101.

FIG. 26 is a diagram for explaining the operation principle of the retraction mechanism 101.

FIG. 27 is a diagram for explaining the operation principle of the retraction mechanism 101.

FIG. 28 is a diagram for explaining an operation example at the time of active retraction.

FIG. 29 is a diagram for explaining the operation example at the time of active retraction.

FIG. 30 is a diagram for explaining an operation example at the time of passive retraction.

FIG. 31 is a diagram for explaining the operation example at the time of passive retraction.

### DESCRIPTION OF EMBODIMENTS

Hereinafter, embodiments of the present disclosure will be described in detail on the basis of the drawings. Note that in each of the following embodiments, the same elements are denoted by the same symbols, and redundant description will be omitted.

The present disclosure will be described in the following order of items.
0. Introduction
1. Embodiment
2. Modifications
3. Exemplary Effects
4. Structure Example of Robot R2 (Support Arm Device)
5. Modification of Robot Device 2
6. Embodiment of Retraction Mechanism
7. Exemplary Effects

### 0. Introduction

For example, in minimally invasive surgery, it is necessary to easily and desirably move an insertion point into a human body of a surgical tool. In a case of a manual procedure, a doctor performs the surgery on the basis of a microscopic image; however, since the insertion point is out of the field of view, the manual procedure is performed while imaging an approximate position. In particular in ophthalmology, there is a risk of causing a tear in a case where a force is applied to an insertion point opened in the sclera (white of the eye). The doctor needs to precisely operate the distal end of the surgical tool in a micrometer order while aligning the position of the insertion point of the surgical tool with a fixed point. Vibration is applied to the eyeball via the insertion point due to shaking of the hand or the like, which causes the target to swing, and thus there is a difficulty that precise operation of the distal end of the surgical tool is hindered. In a case where a surgical robot is used, it is necessary to set the insertion point to the remote center of motion (a fixed point in pivot rotation). It is difficult to accurately align the insertion point with the remote center of motion when the insertion point is moved and stopped.

In the case of the manual procedure, it is necessary for the operator to hold the surgical tool by a hand, and the insertion point and the remote center of motion are manually aligned. In the case of the surgical robot, it is difficult to accurately notify the robot of position information of the insertion point to be set as the remote center of motion. There is a method in which an operator directly touches a robot arm and sets the insertion point by direct teaching. However, an industrial robot is configured as a system in which a force sensor is mounted to a robot arm and passively operates when a person touches the robot arm. This method cannot be used as it is in the medical care due to obstacles of the risk of a runaway of the robot due to a failure of the force sensor or an increase in the size of the arm structure. Alternatively, there is another method of performing force estimation on the basis of a motor current value; however, there is a risk in terms of stability and reliability. In particular, in clinical departments requiring fine work such as microsurgery, a minute positional deviation of several millimeters may cause adverse events in patient tissue.

In some cases, changing the field of view or the like is performed during surgery. At such a time, it is necessary to move the insertion point of the surgical tool. It is necessary to align the insertion point of the surgical tool with the remote center of motion again. For example, it is conceivable to teach the robot accurate three-dimensional position information or to automatically detect the spatial position of the insertion point from image information. However, relative spatial coordinates among the robot, the patient, the camera, and others are required, and it is difficult to improve the reproducibility and accuracy, thereby accompanied by a risk.

According to the disclosed technology, a user such as the doctor can operate the robot by directly applying force by touching the robot or by other means. The robot can be moved or stopped at a desired position, attitude, or the like. It is possible to easily perform alignment of the surgical tool by direct teaching, for example, alignment of the insertion point of the surgical tool and the remote center of motion.

### 1. Embodiment

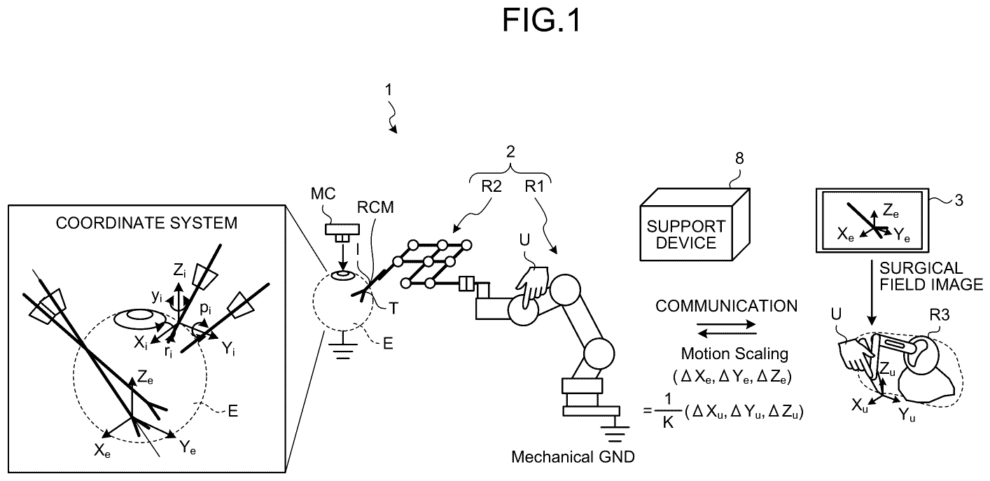

FIG. 1 is a diagram illustrating a schematic configuration example of a robot system 1 according to an embodiment. The robot system 1 is used for surgery. Hereinafter, a case where the surgery is ophthalmic surgery will be described as an example. An eyeball of a patient to be operated is referred to as an eyeball E in the drawing. An operator (doctor or the like) is referred to as a user U in the drawing. In FIG. 1, a hand of the user U is schematically illustrated.

The robot system 1 includes a robot device 2, a microscope MC, a monitor 3, a robot R3, and a support device 8. Describing the microscope MC and the monitor 3 first, the microscope MC observes a surgical field. The field of vision of the microscope MC may include the eyeball E, a surgical tool T in the eyeball E, and others. The monitor 3 displays an observation image (surgical field image) of the microscope MC. The user U observes the surgical field by viewing the observation image of the microscope MC displayed on the monitor 3 or directly viewing from an eyepiece of the microscope MC. An operation using the visual feedback of a relative positional relationship between the surgical tool I captured in the surgical field and the robot device 2 at hand allows the surgery to proceed.

The robot device 2 is a robot (patient-side robot) disposed near a patient and includes two robots connected in series to each other. A first robot is referred to as a robot R1 in the drawing. A second robot is referred to as a robot R2 in the drawing. The robot R1 is positioned farther from the patient than the robot R2 is. The robot R2 is supported by the robot R1 in such a manner as to be positioned closer to the patient than the robot R1 is. The robot device 2 can also be referred to as a support arm device or the like. A base position (base plane) serving as a reference of the spatial coordinates of the robot device 2 is schematically illustrated as Mechanical GND.

The robot R1 is configured in such a manner that the user U can operate the robot R1 by directly applying a force. The robot R1 includes no actuators, no motors, no force sensors, nor the like. The operation of the robot R1 by the user U is also referred to as manual operation of the robot R1. For example, the user U grips and moves the robot R1 to manually operate the robot R1.

The robot R1 has three or more degrees of freedom. In this example, the robot R1 has three degrees of freedom of translation and three degrees of freedom of rotation. In FIG. 1, the translation axes of the robot R1 are indicated as an Xi axis, a Yi axis, and a Zi axis. The rotation axes of the robot R1 are indicated as an ri axis, a pi axis, and a yi axis. By allowing the robot R1 to have many degrees of freedom, it becomes easy to move the robot R1 to a desired position or to make the robot R1 take a desired attitude.

The robot R2 is configured in such a manner that the user U can operate the robot R2 without directly applying a force. The robot R2 includes an actuator and others. For example, the robot R2 is configured to proactively move (driven) in accordance with a displacement amount of the robot R3 provided at a position away from the robot R2. The user U remotely operates the robot R2 by operating the robot R3.

The robot R2 supports the surgical tool T. The surgical tool T is inserted into the body of the patient, in this example, the eyeball E. The robot R2 supports the surgical tool T in such a manner that the surgical tool T has the remote center of motion RCM. In this example, the robot R2 includes a parallel link mechanism, a pivot point of which (pivot position) serves as the remote center of motion RCM.

The robot R2 has one or more degrees of freedom. In this example, the robot R2 has three degrees of freedom and is pivotally movable. In FIG. 1, pivot rotation axes of the robot R2 are indicated as an Xe axis, a Ye axis, and a Ze axis. The robot R2 moves the surgical tool T in the eyeball E about the remote center of motion RCM as a rotation center.

Since the robot R2 is caused to move by a precise actuator or the like, it can perform operation with higher accuracy (for example, about 10 μm) than that of the robot R1 that is manually operated. In this sense, the robot R1 can be referred to as a coarse movement robot, and the robot R2 can be referred to as a fine movement robot. Note that a drape for covering a clean region may be fixed to the robot R1.

The user U manually moves the robot R1 in such a manner as to insert the surgical tool T into the eyeball E. An insertion position of the surgical tool T in the eyeball E is referred to as an insertion point I in the drawing. The user U manually moves the robot R1 to align the insertion point I with the remote center of motion RCM. As illustrated in FIG. 1, the surgery is carried out in a state where the insertion point I of the surgical tool T and the remote center of motion RCM overlap with each other (are at the same position).

The robot R2 is configured to be remotely operable. In the example illustrated in FIG. 1, as described above, the user U remotely operates the robot R2 by operating the robot R3. The axes of the robot R3 corresponding to the robot R2 are illustrated as an Xu axis, a Yu axis, and a Zu axis. The robot R2 and the robot R3 are, for example, bilaterally controlled in such a manner that displacement amounts and forces in the robots R2 and R3 correspond to each other by using bidirectional communication.

Scaling of the relative positional relationship may be performed between the robot R2 and the robot R3. In the example illustrated in FIG. 1, motion scaling is used in such a manner that the physical displacement amount of the robot R2 becomes smaller than the physical displacement amount of the robot R3 (1/K times). This enables fine remote operation of the robot R2 via the robot R3, thereby facilitating remote surgery.

Note that the user U who operates the robot R1 of the robot device 2 and the user U who operates the robot R3 may be the same or different.

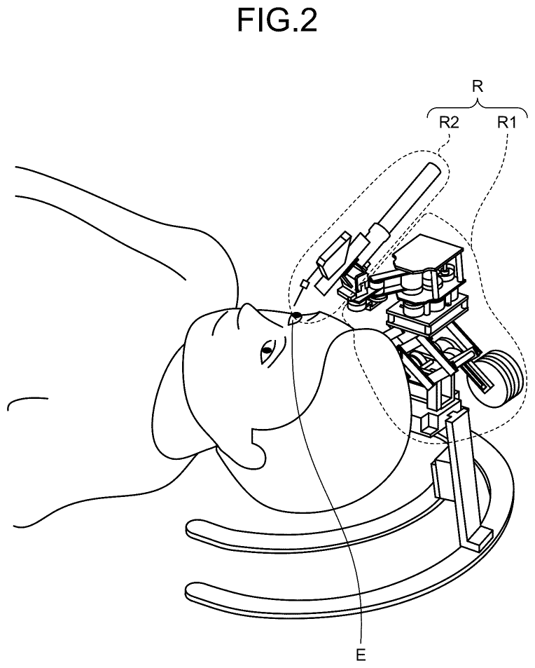

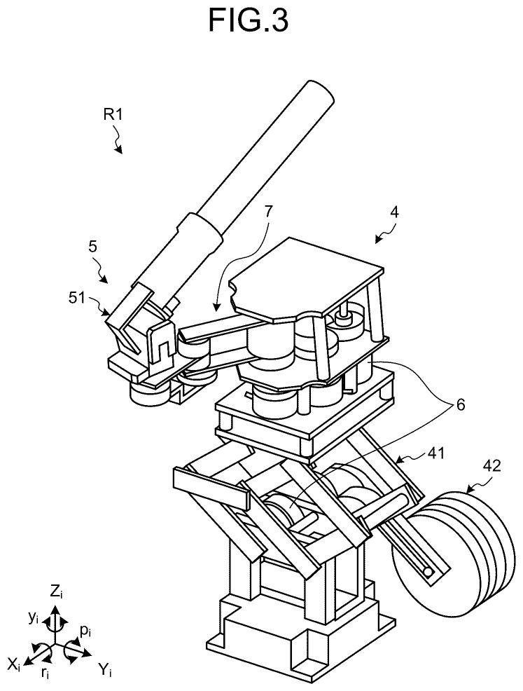

FIG. 2 is a diagram illustrating an example of arrangement of the robot device 2 with respect to the patient. The robot device 2 is arranged such that the robot R1 is fixed to rails each having an arc shape provided at a pedestal near the head of the patient and that the robot R2 is positioned near the eyeball E of the patient. The robot R1 of the robot device 2 will be further described with reference to FIG. 3.

FIG. 3 is a diagram illustrating an example of a schematic structure of the robot R1. The robot R1 includes a base portion 4, a distal end portion 5, lock mechanisms 6, and a transmission 7.

The base portion 4 includes a translation mechanism 41 in order to have a translational degree of freedom. In this example, the translational degree of freedom is three degrees of freedom. The translation mechanism 41 is a parallel link mechanism having translational three degrees of freedom in the vertical direction (Z-axis direction) and the horizontal direction (XY plane direction).

The base portion 4 includes a counterweight 42 in a lower portion. The counterweight 42 improves the balance of the robot R1, thereby providing a dead weight compensation function to the robot R1. For example, provided is such a dead weight compensation function that all axes of the robot device 2 can stay in the place.

The distal end portion 5 supports the robot R1 (FIG. 1). The distal end portion 5 includes a rotation mechanism 51 in order to have a rotational degree of freedom. The rotational degree of freedom may be, for example, two or more, and in this example, the rotational degree of freedom is three degrees of freedom. Examples of the rotation mechanism 51 include a gimbal mechanism, a ball joint mechanism, and others.

The robot R2 may be detachably attached to the distal end portion 5 (for example, the rotation mechanism 51). By attaching and detaching different robots R2 to and from the same robot R1, the robot R1 can be repeatedly used (reused), whereas the robot R2 can be disposable.

The lock mechanisms 6 are provided to the base portion 4 and generate a braking force in such a manner as to lock each joint related to the degrees of freedom of the robot R1. Each joint and a lock mechanism 6 may correspond to each other on a one-to-one basis. Each joint can be individually locked (lock ON) or unlocked (lock OFF). A lock mechanism 6 includes, for example, an electromagnetic brake. The electromagnetic brake may unlock a joint when a current is flowing or a voltage is applied and lock the joint when no current is flowing or no voltage is applied. The joint is locked by turning off a power supply of the lock mechanism 6. The lock mechanism 6 is powered on or off manually by the user U, for example.

The lock mechanism 6 may have a support shaft that rotates depending on the angle of the joint. An angle sensor (potentiometer, encoder, etc.) may be fixed in series to the support shaft. Such an angle sensor makes it possible to detect of the angle of a joint. Since it is not necessary to directly attach the angle sensor to the joint, advantages of miniaturization and weight reduction can be obtained, and the number of electric wires can be reduced. By solving the kinematics using a detection result of the angle sensor (for example, by calculation of forward kinematics), the position and the attitude of the distal end portion 5 from the base portion 4 are calculated. The spatial coordinates of the distal end (arm distal end) of the robot R2 or the surgical tool T with respect to the reference position of the robot device 2 can be calculated.

Each of the lock mechanisms 6 can be individually controlled. The control of the lock mechanism 6 may be performed by the user U, for example, by pedal operation or the like, or may be automatically performed. Locking and unlocking of translational movement of the base portion 4 as well as locking and unlocking of rotational movement of the distal end portion 5 can be controlled separately.

When a lock mechanism 6 is powered off, the lock by the lock mechanism 6 is on. Even when a sudden power failure or the like occurs, the risk of a runaway of the robot device 2 is mitigated or avoided. A lock mechanism 6 by which lock is on also serves as a torque limiter that passively moves when the user U manually and strongly pushes the robot R1. For example, robotic surgery can be switched to a manual procedure in an emergency.

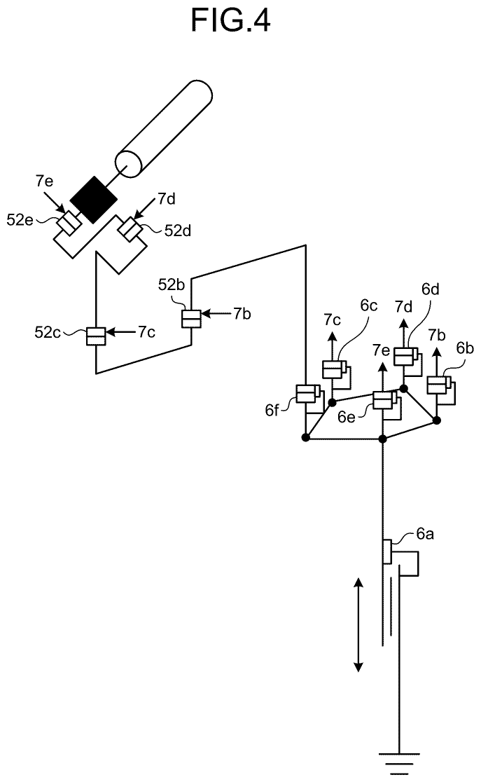

The transmission 7 is provided to the base portion 4 and transmits the braking force from the lock mechanisms 6 to a corresponding joint. This will be described also with reference to FIG. 4.

FIG. 4 is a diagram schematically illustrating an example of transmission of a braking force by the transmission 7. Some joints of the translation mechanism 41 (FIG. 3) of the base portion 4 are directly provided with the lock mechanism 6, and thus transmission of the braking force by the transmission 7 is unnecessary. Such lock mechanisms 6 are exemplified as a lock mechanism 6a and a lock mechanism 6f. Among joints of the translation mechanism 41, to a joint to which no lock mechanism 6 is directly provided, the braking force of a lock mechanism 6 is transmitted via the transmission 7. Such a lock mechanism 6 is exemplified as a lock mechanism 6b. The lock mechanism 6a may be directly attached or may be attached via a speed reducer (or a speed increaser).

No lock mechanism 6 is provided directly at joints of the distal end portion 5, and the braking force of a lock mechanism 6 is transmitted via the transmission 7. The joints of the distal end portion 5 are exemplified as a joint 52c to a joint 52e. Corresponding lock mechanisms 6 are exemplified as a lock mechanism 6c to a lock mechanism 6e. Braking forces of the lock mechanisms 6c to be are transmitted to the joints 52b to 52e via the transmissions 7b to 7e.

For example, by turning on the lock by the lock mechanisms 6c to 6e and turning off the lock by the lock mechanism 6a, the lock mechanism 6b, and the lock mechanism 6f, the base portion 4 can be translated while the rotational movement of the distal end portion 5 is locked. The user U can, for example, directly hold and move or rotate the distal end portion 5.

The transmission 7 does not include a driving force transmission system using a gear. Accordingly, the entire robot R1 can be reduced in size and weight. For example, the transmission 7 transmits the braking force from the lock mechanism 6 to the joints using a wire, a wire rope, a belt, a steel belt, a hydraulic pressure, a pneumatic pressure, a dielectric elastomer, a shape memory alloy, or others. In the example illustrated in FIG. 3, the transmission 7 is a wire transmission that transmits the braking force from the lock mechanisms 6 to the joints using a wire. The wire rope is fixed to joints, and the joints are connected to the lock mechanisms 6 via the wire rope. The locking and unlocking of the three translation axes and the three rotation axes can be switched by the wire-driven system.

Referring back to FIG. 3, the user U grips and translationally moves the base portion 4 of the robot R1 or grips and rotationally moves the distal end portion 5 of the robot R1, thereby manually operating the robot R1. As a result, the user U can move the robot R2 (FIG. 1) to a desired position or stop the robot R2 supported by the distal end portion 5, and eventually, the surgical tool T connected to the robot R2.

Since the robot device 2 is reduced in weight and size, handling of the robot device 2 including manual operation of the robot R1 is facilitated. The robot R1 of the robot device 2 may have a size that can be gripped and operated by the user U with one hand, for example, a palm size of less than or equal to 20 cm. The robot R1 is further downsized than the robot R2 and can have a size of, for example, a tennis ball of less than or equal to 7 cm.

Since the robot R1, which is a coarse movement robot, is small, the scale of the coarse movement is also small, and the vibration noise is reduced. It is known that a resonance frequency corresponding to the vibration noise is inversely proportional to the mass. As the scale of the coarse movement decreases, the mass also decreases, and thus the resonance frequency increases. The vibration noise becomes relatively small. In addition, with a shorter link length, the swing width due to vibration is relatively small.

According to the robot device 2 described above, for example, the user U can easily align the insertion point I of the surgical tool T and the remote center of motion RCM by manually operating the robot R1. Some more specific advantages will be described. For example, since the robot R1 includes no motors nor force sensors, the risks of a runaway or a failure can be reduced.

Since it is possible to reduce the size and weight of the entire robot device 2 (the entire robot arm), it is possible to reduce the force required for the user U to hold and to move the robot device 2 by hand. For example, the operation load becomes light.

The braking forces from the lock mechanisms 6 included in the base portion 4 are transmitted via the transmission 7. Since the lock mechanisms 6 are included in the base portion 4, the structure of the distal end portion 5, that is, the structure on the patient side around the surgical field can be simplified. It is possible to reduce the risk of interference with the surgical tool T at the time of surgery and inhibition of the field of view of the microscope MC and to keep the clean region small, which is highly advantageous in terms of operation. Similar effects can be obtained also from including the transmission 7 in the base portion 4.

Locking and unlocking by the lock mechanisms 6 can be actively switched. The need for the user U to spend a lot of time for moving the insertion point I of the surgical tool T that needs to be frequently performed during surgery is reduced.

For example, a macro positioning arm disclosed in Patent Literature 1 has a structure including a degree of freedom of electric power. In this case, a motor, an encoder, a force sensor, and the like are required for controlling the degree of freedom of electric power, the device is increased in size with an increased weight, and the manufacturing cost is also increased. In addition, the arm of Patent Literature 1 uses a rack-and-pinion transmission and thus has a problem of backlash. Since the weight is structurally increased, the actuator output is increased. For example, such a problem is addressed by the robot device 2 according to the embodiment.

The lock mechanisms 6 are included in the base portion 4 away from the patient, rather than on the distal end portion 5 positioned near the patient. The distal end portion 5 can be made compact, whereby it is made possible to prevent problems such as interference with another surgical tool T, occlusion that blocks the surgical field, or contact with the patient.

The support device 8 will be described with reference to FIG. 1 again. The support device 8 supports the operation of the robot R1 by the user U for aligning the insertion point I of the surgical tool T and the remote center of motion RCM. The support device 8 may be implemented by, for example, operating software on a general-purpose computer, or may be implemented by dedicated hardware. The support device 8 acquires necessary information from other elements of the robot system 1 by communication or the like. For example, information regarding the state of the robot device 2, a captured image of the microscope MC, and others are acquired.

The support device 8 notifies the user U of the positional deviation between the insertion point I of the surgical tool T and the remote center of motion RCM. The notification method is not particularly limited, and, for example, display by a display included in the support device 8, sound output by a speaker, or others may be used. As the display, the monitor 3 may be used.

The support device 8 detects a positional deviation between the insertion point I and the remote center of motion RCM. Some examples of the detection method will be described.

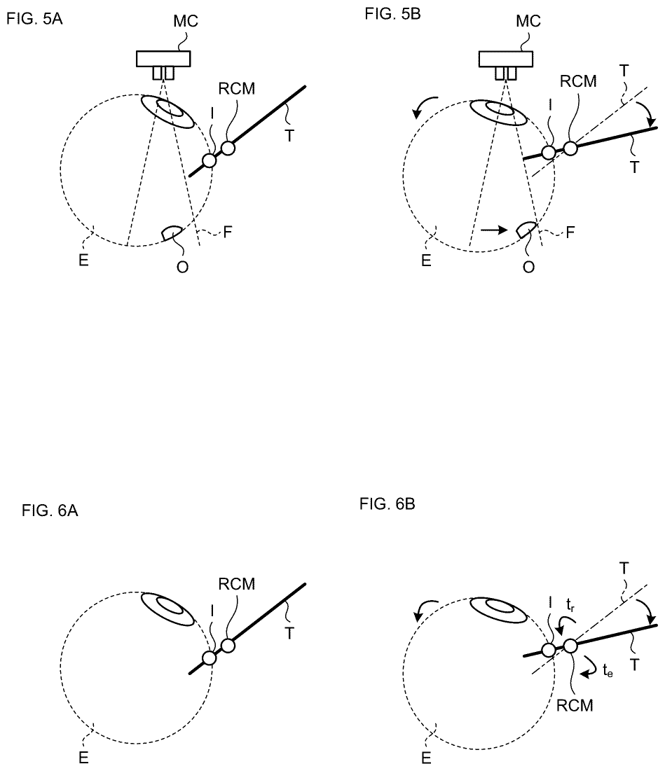

The support device 8 may detect a positional deviation between the insertion point I of the surgical tool T and the remote center of motion RCM on the basis of a change in observation images of the microscope MC at the time when the surgical tool T rotates. This will be described with reference to FIG. 5.

FIG. 5 is a diagram illustrating an example of detection of a positional deviation between the insertion point I of the surgical tool T and the remote center of motion RCM. As illustrated, the position of the remote center of motion RCM is shifted from the position of the insertion point I. An object O (for example, some biological tissue) is located in the field of view F of the microscope MC, and the observation image includes the object O. When the surgical tool T rotates as indicated by an arrow in (B) of FIG. 5, the insertion point I moves due to the positional deviation between the insertion point I and the remote center of motion RCM, and the eyeball E rotates. The position of the object O in the observation image of the microscope MC also moves. By detecting this movement, the positional deviation between the insertion point I and the remote center of motion RCM is detected.

The support device 8 may detect a positional deviation between the insertion point I of the surgical tool T and the remote center of motion RCM on the basis of a reaction force from the insertion point I of the surgical tool T upon the rotation of the surgical tool T. This will be described with reference to FIG. 6.

FIG. 6 is a diagram illustrating an example of detection of a positional deviation between the insertion point I of the surgical tool T and the remote center of motion RCM. As illustrated, the position of the remote center of motion RCM of the surgical tool T is shifted from the position of the insertion point I. As indicated by an arrow in (B) of FIG. 6, when the surgical tool T rotates, the insertion point I moves due to the positional deviation between the insertion point I and the remote center of motion RCM, and the eyeball E rotates. Accordingly, action torque the and reaction torque tᵣ are generated. The reaction torque tᵣ is based on a reaction force from the insertion point I applied to a drive shaft when the surgical tool T rotates. By detecting generation of the reaction torque tᵣ by, for example, measuring or estimating, movement of the insertion point I, that is, the positional deviation between the insertion point I and the remote center of motion RCM is detected. For example, a detection result of a resistance value of an actuator provided at a joint of the robot R2 may be used.

### 2. Modifications

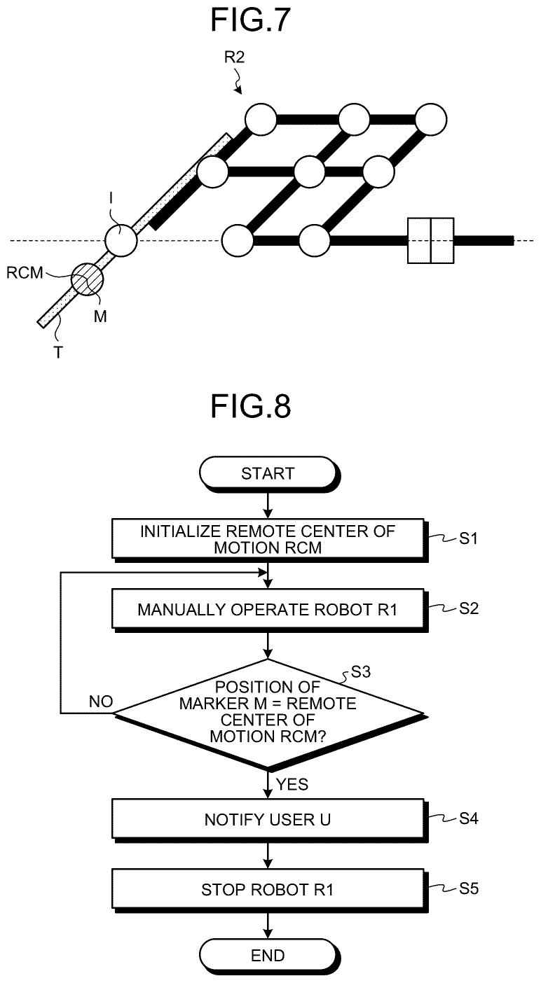

The technology disclosed is not limited to the above embodiment. Some modifications will be described. For example, a marker may be provided at a position corresponding to the remote center of motion RCM of the surgical tool T (the surgical tool I may be marked). This will be described with reference to FIG. 7.

FIG. 7 is a diagram illustrating an example of marking of the surgical tool T. The surgical tool T has a marker M. The marker M is a physical marker provided at a position corresponding to the remote center of motion RCM of the surgical tool T. The marker M may be, for example, one that can be recognized (visually recognized or the like) by the user U or one that can be recognized by the support device 8 on the basis of a captured image of a camera or the like included in the support device 8. For example, the marker M may have a color, a shape, or the like that is different from those of other portions of the surgical tool T.

The user U can manually operate the robot R1 while confirming the position of the marker M. This facilitates alignment between the insertion point I of the surgical tool T and the remote center of motion RCM. Furthermore, the support device 8 may notify the user U that the position of the marker M coincides with the remote center of motion RCM. This will be described with reference to FIG. 8.

FIG. 8 is a flowchart illustrating an example of alignment between the insertion point I of the surgical tool T and the remote center of motion RCM using the marker M.

In Step S1, the remote center of motion RCM is initialized. The position of the remote center of motion RCM in the surgical tool T in a state where the surgical tool T is connected to the robot R2 is grasped by the support device 8. For example, the position of the remote center of motion RCM in the captured image of the camera included in the support device 8 is registered.

In Step S2, the user U manually operates the robot R1. For example, first, the user U manually operates the robot R1 so that the remote center of motion RCM is positioned outside the eyeball E (for example, so as to be in a state immediately before insertion). Then, the user U manually operates the robot R1 in such a manner as to insert the surgical tool T into the eyeball E. The marker M of the surgical tool T (namely, the remote center of motion RCM) approaches the insertion point I. In the support device 8, the positional relationship between the insertion point I of the surgical tool T and the marker M is monitored.

In Step S3, the support device 8 determines whether the position of the marker M coincides with the remote center of motion RCM. If they coincide with each other (Step S3: Yes), the processing proceeds to Step S4. If not (Step S3: No), the processing returns to Step S2.

In Step S4, the support device 8 notifies the user U that the insertion point I of the surgical tool T matches the remote center of motion RCM.

In Step S5, the user U stops the robot R1. For example, all the joints are locked by the lock mechanisms 6. The robot R1 is fixed in a state where the insertion point I of the surgical tool T and the remote center of motion RCM are aligned.

For example, as in the above manner, the marker M provided to the surgical tool T and, furthermore, the support device 8 can be used to support the alignment of the insertion point I of the surgical tool T and the remote center of motion RCM by the user U.

In the above embodiment, a case where the surgery is ophthalmic surgery has been described as an example. However, the disclosed technology may be applied to surgery other than ophthalmic surgery.

### 3. Exemplary Effects

The technology described above is specified as follows, for example. One piece of the disclosed technology is the robot device 2. The robot device 2 includes the robot R1 (first robot) and the robot R2 (second robot) as described by referring to FIGS. 1 to 3. The robot R1 includes a base portion 4 and a distal end portion 5. The robot R2 is supported by the distal end portion 5 of the robot R1 and supports the surgical tool T inserted into the body of the patient (for example, into the eyeball E). The robot R1 is configured such that the user U directly applies a force to the robot R1 to perform an operation (manual operation).

According to the robot device 2 described above, the user U can easily perform alignment of the surgical tool T by manually operating the robot R1.

As described with reference to FIG. 3 and others, the robot R1 may include the lock mechanisms 6 that lock the joints. This enables switching between locking and unlocking of the joints. For example, it is possible to reduce the need for the user U to spend a lot of time for moving the insertion point I of the surgical tool T that needs to be frequently performed during surgery.

As described with reference to FIG. 3 and others, a lock mechanism 6 may include an electromagnetic brake that unlocks a joint when a voltage is applied and locks the joint when no voltage is applied. As a result, it is possible to reduce the risk of a runaway of the robot device 2 caused by, for example, occurrence of a power failure.

As described with reference to FIG. 3 and others, the lock mechanisms 6 may be included in the base portion 4. This can simplify the structure of the distal end portion 5.

As described with reference to FIGS. 3, 4, and others, the robot R1 includes the transmission 7 that transmits the braking force from the lock mechanisms 6 to the joints, and the transmission 7 may transmit the braking force from the lock mechanisms 6 to the joints using at least one of a wire, a wire rope, a belt, a steel belt, a hydraulic pressure, a pneumatic pressure, a dielectric elastomer, or a shape memory alloy. This makes it possible to reduce the size and the weight of the device as compared with, for example, a case where a driving force transmission system using a gear is used.

As described with reference to FIG. 3 and others, the transmission 7 may be included in the base portion 4. This can simplify the structure of the distal end portion 5.

As described with reference to FIGS. 1, 3, and others, the base portion 4 may have translational three degrees of freedom, and the distal end portion 5 may have rotational two or more degrees of freedom. For example, by allowing the robot R1 to have many degrees of freedom by the base portion 4 and the distal end portion 5 described above, it is made possible to easily move the robot R1 to a position or make the robot R1 to have a desired attitude.

As described with reference to FIG. 3 and others, the robot R2 may be detachably attached to the distal end portion 5 (for example, the rotation mechanism 51) of the robot R1. As a result, the robot R1 can be repeatedly used, and the robot R2 can be made disposable.

As described with reference to FIG. 1 and others, the robot R2 may be configured to be remotely operable. This enables remote surgery.

As described with reference to FIG. 7 and others, the surgical tool T may have the marker M physically provided at a position corresponding to the remote center of motion RCM of the surgical tool T. This facilitates alignment between the insertion point I of the surgical tool T and the remote center of motion RCM.

As described with reference to FIGS. 1, 3, and others, the robot R1 may have a size that can be gripped and operated by the user U with one hand, and the robot R2 may be smaller than the robot R1. With the robot device 2 configured by such small-sized robot R1 and robot R2, it is made possible to facilitate handling of the robot device 2 including manual operation of the robot R1.

The robot system 1 described with reference to FIGS. 1, 5 to 8, and others is also one piece of the disclosed technology. The robot system 1 includes the robot device 2 and the support device 8 described above. The support device 8 supports the operation of the robot R1 by the user U for aligning the insertion point I of the surgical tool T and the remote center of motion RCM of the surgical tool T. For example, the support device 8 notifies the user U of the positional deviation between the insertion point I of the surgical tool T and the remote center of motion RCM. In that case, the support device 8 may detect a positional deviation between the insertion point I of the surgical tool T and the remote center of motion RCM on the basis of a change in observation images of the microscope MC observing the surgical field at the time when the surgical tool T rotates. Alternatively, the support device 8 may detect a positional deviation between the insertion point I of the surgical tool T and the remote center of motion RCM on the basis of a reaction force from the insertion point I of the surgical tool T upon the rotation of the surgical tool T. As a result, it is possible to align the insertion point I of the surgical tool T more easily with the remote center of motion RCM.

As described with reference to FIGS. 7, 8, and others, the support device 8 may notify the user U that the positions of the insertion point I of the surgical tool T and the marker M physically provided at the position corresponding to the remote center of motion RCM in the surgical tool T coincide with each other. It is also possible to support the operation of the robot R1 by the user U in this manner.

The disclosed technology can also be specified as follows. For example, the robot system 1 is an ophthalmic surgery supporting robot system and may include a fine movement robot (robot R2) and a coarse movement robot arm (robot R1) that supports the fine movement robot (robot R2) in series at the distal end portion 5.

The position and the attitude of the coarse movement robot arm may be manually moved and stopped by the operator (user U). The coarse movement robot arm may have an ON/OFF mechanism (for example, the lock mechanisms 6) for the joint lock. The coarse movement robot arm may have a dead weight compensation function (for example, the counterweight 42 provided in the base portion 4). The coarse movement robot arm may be a passive robot arm having at least three or more degrees of freedom of position and attitude. The coarse movement robot arm may have a small and light-weighted structure. The joint rotation angle of the coarse movement robot arm may be measured by a sensor, and the position and the attitude of the arm distal end from the base may be calculated by solving kinematics. The coarse movement robot arm may have a rotational degree of freedom at the distal end portion (e.g. the distal end portion 5) by a gimbal or a ball joint (e.g. the rotation mechanism 51).

The fine movement robot may be an active robot arm having a mechanical remote center of rotation. The robot R2 may have at least one or more degrees of freedom, include a remote center of rotation (remote center of motion RCM), and be actively driven. The robot R2 may have a small and light-weighted structure.

Note that the effects described herein are merely examples, and it is not limited to the disclosed content. There may be other effects.

### 4. Structure Example of Robot R2 (Support Arm Device)

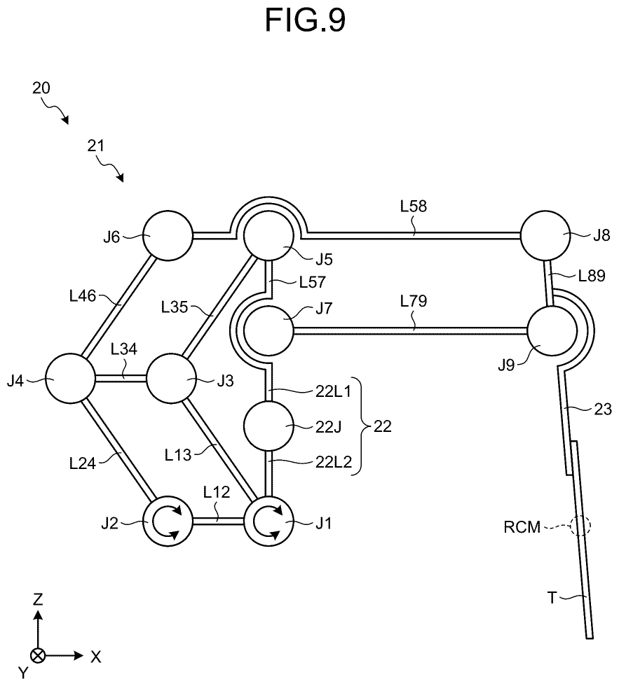

As described earlier, the robot R2 has the parallel link mechanism. A slider mechanism that linearly moves the surgical tool in the insertion direction from the base of the parallel link mechanism may be connected to the parallel link mechanism. A structure not using a slider mechanism is also possible. The robot R2 capable of linearly moving the surgical tool in the insertion direction from the base of the parallel link mechanism without using the slider mechanism is referred to as a support arm device 20, which will be described with reference to FIGS. 9 to 13. Note that the coordinate system regarding the support arm device 20 illustrated in FIGS. 9 to 13 may be defined separately from the coordinate system regarding the robot R2 illustrated in FIG. 1 and others described earlier.

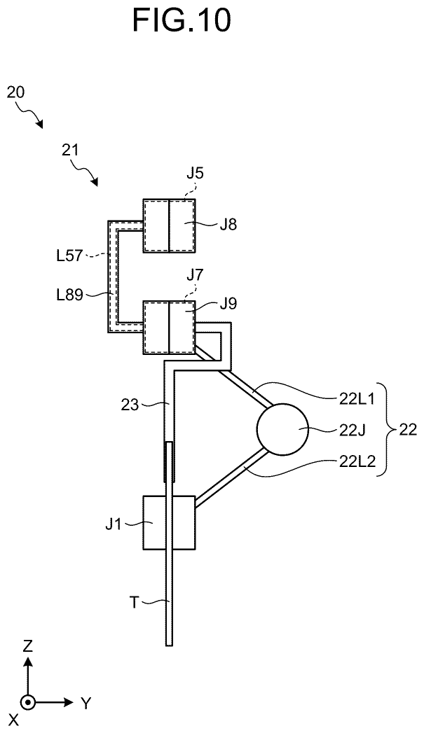

FIGS. 9 and 10 are diagrams schematically illustrating an example of a schematic structure of the support arm device 20. In FIG. 9, an XYZ coordinate system is illustrated. The X-axis direction, the Y-axis direction, and the Z-axis direction correspond to a front-rear direction, a lateral direction, and an up-down direction of the support arm device 20. In FIG. 9, the schematic structure of the support arm device 20 as viewed from a side (in the Y-axis positive direction) is schematically illustrated. In FIG. 10, some elements of the support arm device 20 as viewed from the front (in the X-axis negative direction) are schematically illustrated. An element indicated by a broken line is positioned behind an element indicated by a solid line (on the X-axis negative direction side).

As illustrated in FIG. 9, the support arm device 20 supports the surgical tool T on the distal end side (X-axis positive direction side). The support arm device 20 includes a parallel link mechanism 21, a connection mechanism 22, and a support member 23.

The parallel link mechanism 21 extends in the XZ plane direction (plane direction of a first plane). That is, the parallel link mechanism 21 has two degrees of freedom of a degree of freedom in the X-axis direction and a degree of freedom in the Z-axis direction. There is no degree of freedom in the Y-axis direction.

The parallel link mechanism 21 includes a plurality of joints and a plurality of links. In FIG. 9, joints J1 to J9 are illustrated as the plurality of joints of the parallel link mechanism 21. Among them, the joint J1 and the joint J2 are arranged at the base of the parallel link mechanism 21 (of the support arm device 20). The base of the parallel link mechanism 21 is an end on the opposite side to the distal end side of the parallel link mechanism 21. The joint J1 and the joint J2 are rotationally driven by an actuator (not illustrated) or the like. Mechanical parts such as the actuator are intensively arranged at the base of the parallel link mechanism 21.

Each of the plurality of links extends in the XZ plane direction and is connected between joints. In the example illustrated in FIG. 9, a link L12 is connected between the joint J1 and the joint J2. A link L13 is connected between the joint J1 and the joint J3. A link L24 is connected between the joint J2 and the joint J4. A link L34 is connected between the joint J3 and the joint J4. A link L35 is connected between the joint J3 and the joint J5. A link L46 is connected between the joint J4 and the joint J6. A link L57 is connected between the joint J5 and the joint J7. A link L58 is connected between the joint J5 and the joint J8. A link L79 is connected between the joint J7 and the joint J9. A link L89 is connected between the joint J8 and the joint J9.

Note that, in FIG. 9, portions of some links positioned at positions different from other elements in the Y-axis direction are drawn in such a manner as to bypass a joint.

The parallel link mechanism 21 includes three parallel link mechanisms including a parallel link mechanism positioned on the base side, a parallel link mechanism positioned on the distal end side, and a parallel link mechanism connected therebetween. The parallel link mechanism positioned on the base side includes the joints J1 to J4, the link L12, the link L13, the link L24, and the link L34. The parallel link mechanism positioned on the distal end side includes the joint J5, joints J7 to J9, the link L57, the link L58, the link L79, and the link L89. The parallel link mechanism connected between them includes the joints J3 to J6, the link L34, the link L35, the link L46, and the link L58.

The operation of the illustrated parallel link mechanism 21 itself will be understood by those skilled in the art, and thus several characteristic portions of the parallel link mechanism 21 will be described below.

The link L89 is a link (support link) that supports the surgical tool T on the distal end side. In this example, the link L89 is connected to the surgical tool T via the support member 23 and supports the surgical tool T. The link L89, the support member 23, and the surgical tool T extend in the insertion direction of the surgical tool T into the body. The link L57 is a link (opposing link) facing the link L89 and extends in the insertion direction of the surgical tool T similarly to the link L89. In the insertion direction of the surgical tool T, the surgical tool T, the link L89, and the link L57 move together in parallel.

The joint J5 is a joint (first joint) connected to a first end of the link L57. The joint J7 is a joint (second joint) connected to a second end of the link L57.

As described above, the joint J1 is a joint (third joint) that is disposed at the base of the parallel link mechanism 21 together with the joint J2 and is rotationally driven. By rotating the joint J1 and the joint J2, the surgical tool T can be moved in the XZ plane direction from the base of the parallel link mechanism 21. For example, the surgical tool T can be pivoted or moved in the insertion direction by rotation from the base.

The support arm device 20 supports the surgical tool T in such a manner that the surgical tool T has the remote center of motion RCM. Specifically, the support arm device 20 supports the surgical tool T in such a manner that an intersection between a straight line connecting the joint J1 and the joint J2 and the surgical tool T is set to the remote center of motion RCM. In the example illustrated in FIG. 9, the remote center of motion RCM of the surgical tool T is positioned at the same position as the joint J1 and the joint J2 in the Z-axis direction.

The connection mechanism 22 is connected to joints of the parallel link mechanism 21 in such a manner as to linearly move the surgical tool I in the insertion direction from the base of the parallel link mechanism 21. In this example, the connection mechanism 22 is connected between the joint J7 and the joint J1. The connection mechanism 22 is transformed in the plane direction of a second plane intersecting an XZ plane in such a manner that the joint J7 moves relative to the joint J1 in the extending direction of the link L57 (namely, the insertion direction of the surgical tool T). Hereinafter, unless otherwise specified, it is based on the premise that the second plane is also a YZ plane orthogonal to an XZ plane.

The connection mechanism 22 is transformed on the YZ plane in such a manner that the joint J5, the joint J7, the joint J1, and the connection mechanism 22 are positioned on the YZ plane. That is, the connection mechanism 22 is transformed in such a manner that the YZ plane passing through the joint J5, the joint J7, and the joint J1 is restrained.

In one embodiment, the connection mechanism 22 includes a link mechanism that pivots on the YZ plane. The surgical tool T moves in the insertion direction as the connection mechanism 22 is transformed. The movement amount of the surgical tool I also changes in correspondence to the transformation amount of the connection mechanism 22.

In the example illustrated in FIGS. 9 and 10, as particularly illustrated in FIG. 10, the connection mechanism 22 includes a link mechanism that is transformed to have a V-shape on the YZ plane. Examples of elements of the link mechanism of the connection mechanism 22 include a joint 22J, a link 22L1, and a link 22L2. The link 22L1, the joint 22J, and the link 22L2 are connected in this order between the joint J7 and the joint J1.

As the joint 22J moves away from the joint J1 and the joint J2, the joint J7 moves to approach the joint J1. In the example illustrated in FIG. 10, as the joint J2 advances in the Y-axis positive direction, the joint J7 moves downward, namely, in the insertion advancing direction of the surgical tool T. The link L57 and the joint J5 also move in the same direction together with the joint J7, and the link L89 facing the link L57 also moves in the same direction. The surgical tool T supported by the link L89 via the support member 23 moves in the insertion advancing direction.

Conversely, as the joint 22J approaches the joint J1 and the joint J7, the joint J7 moves away from the joint J1. In the example illustrated in FIG. 10, as the joint J2 advances in the Y-axis negative direction, the joint J7 moves upward, namely, in the insertion retraction direction of the surgical tool T. The link L57 and the joint J5 also move in the same direction together with the joint J7, and the link L89 facing the link L57 also moves in the same direction. The surgical tool T supported by the link L89 via the support member 23 moves in the insertion retraction direction.

For example, by using transformation of the connection mechanism 22 as described above, the joint J7 and the link L57 can be moved in parallel with the insertion direction of the surgical tool T. As a result, the surgical tool T can be linearly moved in the insertion direction from the base of the parallel link mechanism 21.

As described above, since the parallel link mechanism 21 has no degree of freedom in the Y-axis direction, the joint J5, the joint J7, and the joint J1 do not move in the Y-axis direction even when the connection mechanism 22 is transformed. In addition, since the connection mechanism 22 is transformed not on the XZ plane but on the YZ plane, the joint J5, the joint J7, and the joint J1 do not move in the X-axis direction even when the connection mechanism 22 is transformed. As a result, the three joints of the joint J5, the joint J7, and the joint J1 pass on the same straight line in plan view on the XZ plane, and the joint J5, the joint J7, the joint J1, and the connection mechanism 22 are positioned on the YZ plane. With this condition satisfied, the intersection between the straight line connecting the joint J1 and the joint J2 and the surgical tool T is set as the remote center of motion RCM.

According to the support arm device 20 described above, the surgical tool I can be linearly moved in the insertion direction from the base of the parallel link mechanism 21 in which the rotationally driven joint J1 is disposed by using the transformation of the connection mechanism 22. For example, no large sliding friction is generated as in a slider mechanism. There is also an enhanced possibility of simplifying the structure, facilitating downsizing, or reducing inertia.

In addition, the support arm device 20 can be made compact by folding the connection mechanism 22 (in the above case, the link mechanism thereof). Furthermore, the movable range in the insertion direction of the surgical tool I can be easily expanded as compared with the case of using a slider mechanism. This is because, in the case of a slider mechanism, it is necessary to take measures such as lengthening the slider; however, this difficult in many cases due to size restriction or others.

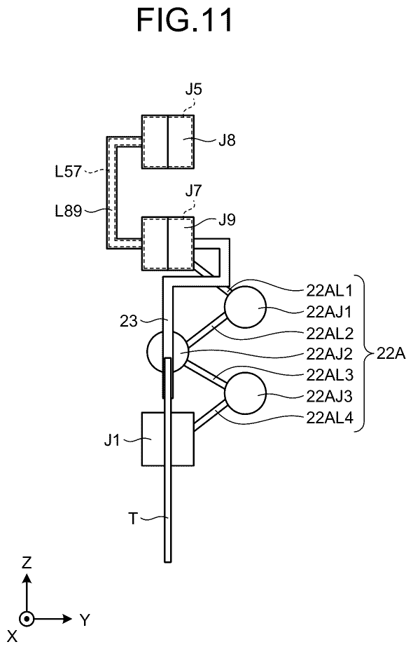

Some modifications of the connection mechanism 22 will be described with reference to FIGS. 11 to 13.

FIG. 11 is a diagram schematically illustrating an example of a schematic structure of a connection mechanism 22A according to a modification. The illustrated connection mechanism 22A includes a link mechanism that is transformed to have a plurality of V-shapes on the YZ plane.

Examples of elements of the link mechanism of the connection mechanism 22A include a joint 22AJ1, a joint 22AJ2, a joint 22AJ3, a link 22AL1, a link 22AL2, a link 22AL3, and a link 22AL4. The link 22AL1, the joint 22AJ1, the link 22AL2, the joint 22AJ2, the link 22AL3, the joint 22AJ3, and the link 22AL4 are connected in this order between the joint J7 and the joint J1.

In the Z-axis direction, the joints positioned on the Y-axis positive direction side and the joint positioned on the Y-axis negative direction side are alternately arranged. In this example, among the joint 22AJ1, the joint 22AJ2, and the joint 22AJ3, the joint 22AJ1 and the joint 22AJ3 are positioned on the Y-axis positive direction side. The joint 22AJ2 is positioned on the Y-axis negative direction side. With such joints and links, transformation to have the plurality of V-shapes is made possible. It is made possible to fold more compactly than in the case of transformation to have the one V-shape (FIG. 10). The possibility of further expanding the movable range is also increased.

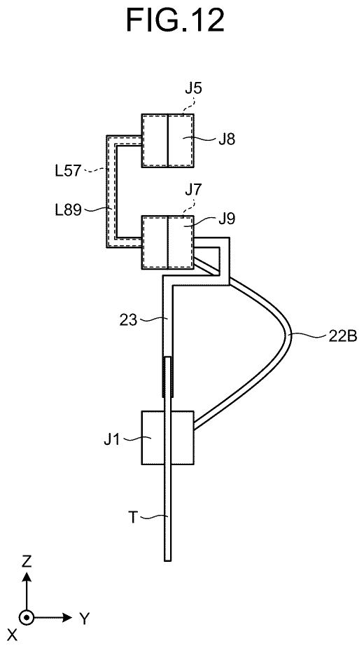

FIG. 12 is a diagram schematically illustrating an example of a schematic structure of a connection mechanism 22B according to a modification. The illustrated connection mechanism 22B includes an elastic body that is elastically transformed on the ZY plane. An example of the elastic body is a leaf spring or the like. The connection mechanism 22B includes a leaf spring that is transformed to have a U-shape on the ZY plane. The connection mechanism 22B also functions similarly to the connection mechanism 22 (FIG. 10) and the connection mechanism 22A (FIG. 11) described above.

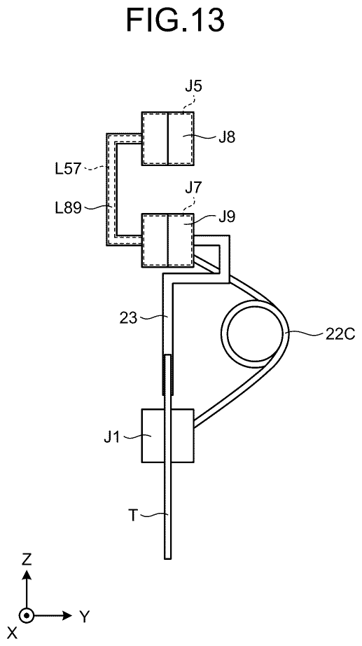

FIG. 13 is a diagram schematically illustrating an example of a schematic structure of a connection mechanism 22C according to a modification. The illustrated connection mechanism 22C includes an elastic body that is transformed in such a manner as to spiral one or more times on the YZ plane. The connection mechanism 22C also functions similarly to the connection mechanism 22 (FIG. 10) and the connection mechanism 22A (FIG. 11) described above. Note that the elastic body may be transformed in such a manner as to be wound or unwound on the YZ plane.

In the above embodiment, the case where the second plane intersecting the XZ plane (first plane) is the YZ plane orthogonal to the XY plane has been described as an example. However, the second plane may not be orthogonal to the XY plane. Various planes other than the XY plane may be the second plane.

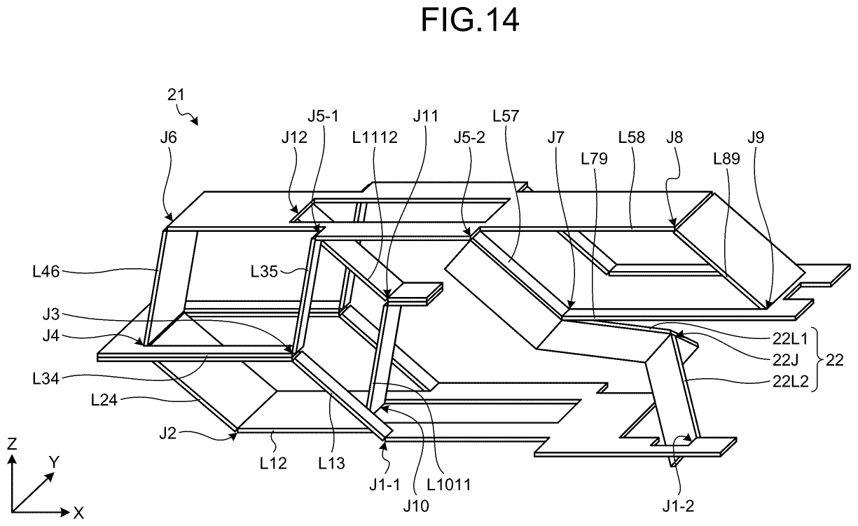

The parallel link mechanism 21 and the connection mechanism 22 can also be assembled in an origami fashion. Such modifications will be described with reference to FIGS. 14 to 17.

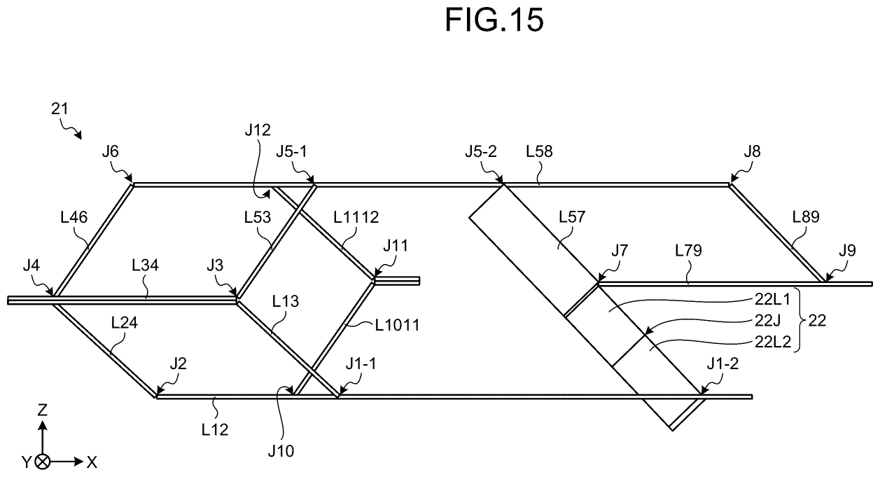

FIGS. 14 and 15 are diagrams illustrating an example of a schematic structure of a parallel link mechanism 21 and a connection mechanism 22 according to the modification. The parallel link mechanism 21 and the connection mechanism 22 are structured using a bendable plate-shaped member in such a manner as to have a hinge structure. A bent portion in the plate-shaped member functions as a joint. A portion connecting bent portions functions as a link. Hereinafter, a portion corresponding to a joint in the plate-shaped member is also simply referred to as a joint portion or the like. A portion corresponding to a link in the plate-shaped member is also simply referred to as a link portion or the like.

A joint portion of the plate-shaped member has flexibility and is elastically transformable (for example, has a hinge structure). A joint portion is softer than a link portion. In other words, a link portion has higher rigidity than that of a joint portion.

For example, the thickness of a joint portion may be less than the thickness of a link portion. The joint portions may have one or more holes (micropores). With a thin thickness or holes included, the joint portions are softer than the link portions and are easily bent.

Examples of the material of the plate-shaped member include carbon, iron, and others. In an embodiment, the plate-shaped member may be made of a composite material. In this case, different materials may be used for joint portions and link portions. A joint portion is made of a material softer than that of a link portion (for example, a material having a different Young's modulus or the like). Examples of such a material of the joint portions include polyimide, rubber, silicone, elastomer, and the like.

In this example, a joint J1 includes a joint J1-1 and a joint J1-2 positioned at different positions in the X-axis direction. The joint J1-2 is positioned on a side opposite to the joint J1 across the joint J1-1. The joint J1-1 and a joint J2 are drive axes, and an intersection between a straight line connecting the joint J1-1 and the joint J2 and a surgical tool T (FIG. 1 and others) is set as the remote center of motion RCM. A connection mechanism 22 is connected between the joint J1-2 and a joint J7. The position of the joint J1-1 may be any position between the joint J1-2 and the joint J2.

In this example, the joint J5 is constituted by a joint J5-1 and a joint J5-2 positioned at different positions in the X-axis direction. The joint J5-2 is positioned on the side opposite to a joint J6 across the joint J5-1. The joint J5-2 is a joint (first joint) connected to a first end of the link L57. As described above, a link L57 is a link (opposing link) facing a link L89 and moves in parallel to and together with the link L89. The position of the joint J5-1 may be any position between the joint J5-2 and the joint J6.

A portion between the joint J1-2 in the parallel link mechanism 21 and the joint J2 and a portion between the joint J5-2 and the joint J6 are connected. As elements used for this connection, a joint J10, a joint J11, a joint J12, a link L1011, and a link L1112 are indicated as an example.

The joint J10 is provided between the joint J12-1 and the joint J2. The joint J12 is provided between the joint J5-2 and the joint J6. The link L1011, the joint J10, and the link L1112 are connected in this order between the joint J11 and the joint J12. These elements are provided from the viewpoint of bonding plate-shaped members described later and do not hinder movement of the parallel link mechanism 21.

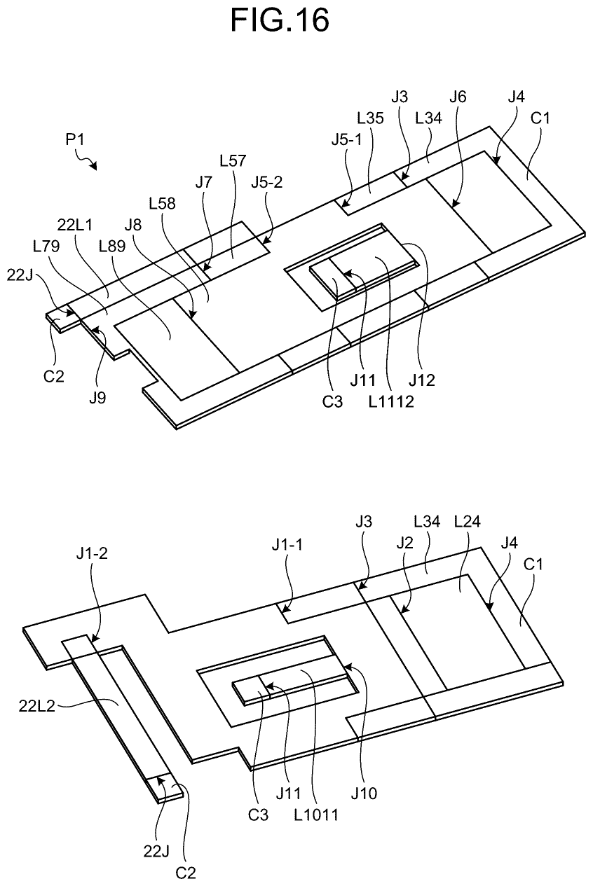

The plate-shaped members forming the parallel link mechanism 21 and the connection mechanism 22 are a plurality of plate-shaped members partially bonded to each other. As an example, a structure in which the parallel link mechanism 21 and the connection mechanism 22 are assembled by bonding two plate-shaped members will be described with reference to FIGS. 16 and 17.

FIG. 16 is a diagram illustrating an example of assembly of the parallel link mechanism 21 and the connection mechanism 22. Two plate-shaped members of a plate-shaped member P1 and a plate-shaped member P2 are used. The plate-shaped member P1 corresponds to an upper side (Z-axis positive direction side) portion of the parallel link mechanism 21 and the connection mechanism 22. The plate-shaped member P2 corresponds to a lower side (Z-axis negative direction side) portion of the parallel link mechanism 21 and the connection mechanism 22. The joints and links corresponding to the plate-shaped member P1 and the plate-shaped member P2 are as indicated by reference numerals in FIG. 16.

Each of the plate-shaped member P1 and the plate-shaped member P2 includes a bonding portion C1, a bonding portion C2, and a bonding portion C3. A bonding portion C1 is connected to a joint J4. A bonding portion C2 is connected to a joint 22J. A bonding portion C3 is connected to the joint J11.

FIG. 17 is a diagram illustrating an example of the schematic structure of the parallel link mechanism 21 and the connection mechanism 22 that are assembled. In a state where each portion of the plate-shaped member P1 and the plate-shaped member P2 are bent, the bonding portions C1 to C3 of each of the plate-shaped member P1 and the plate-shaped member P2 are bonded to each other. In this example, the bonding portion C1, the bonding portion C2, and the bonding portion C3 of the plate-shaped member P1 are bonded to the bonding portion C1, the bonding portion C2, and the bonding portion C3 of the plate-shaped member P2, respectively, in a state where the bonding portions C1, C2, and C3 are in surface contact with each other. The bonding means is not particularly limited. For example, an adhesive agent or the like may be used.

The support arm device 20 including the parallel link mechanism 21 and the connection mechanism 22 as described above is specified as follows, for example. As described with reference to FIG. 14, FIG. 15, and others, the support arm device 20 includes the bendable plate-shaped members forming the parallel link mechanism 21 and the connection mechanism 22, the bent portions (joint portions) of the plate-shaped members function as joints, and a portion (link portion) connecting bent portions in the plate-shaped members functions as a link.

Also in the support arm device 20, as described above, the surgical tool T can be linearly moved in the insertion direction from the base of the parallel link mechanism 21 in which the rotationally driven joint J1-1 is disposed by using the transformation of the connection mechanism 22. In addition, the thickness of the links can be reduced by using the plate-shaped members. Accordingly, for example, the operating area of the parallel link mechanism 21 expands. It is also possible to reduce the weight of the entire parallel link mechanism 21 and the entire connection mechanism 22. Since the function of a joint is implemented by a bent portion of the plate-shaped members, it is possible to avoid rattling that may occur in a case where, for example, a bearing or the like. Since no backlash occurs, the control accuracy of the rotational position can be improved accordingly.

A bent portion (joint portion) of the plate-shaped members is elastically transformable, and a portion (link portion) connecting bent portions in the plate-shaped members may have higher rigidity than that of a bent portion. For example, the functions of a joint and a link can be implemented using such plate-shaped members.

As described with reference to FIG. 16, FIG. 17, and others, the plate-shaped members may be a plurality of plate-shaped members (for example, the plate-shaped member P1 and the plate-shaped member P2) in which parts thereof (for example, the bonding portion C1, the bonding portion C2, and the bonding portion C3) are bonded to each other. The parallel link mechanism 21 and the connection mechanism 22 can be easily manufactured only by bonding the plate-shaped members.

FIG. 18 is a diagram illustrating an example of a schematic structure of a robot R1 and a robot R2. The robot R2, which is the support arm device 20 including the plate-shaped members as described above, is used while being supported by the distal end portion 5 of the robot R1.

### 5. Modification of Robot Device 2

In the above embodiment, the case where the robot R1 has six degrees of freedom, namely, translational three degrees of freedom in the base portion 4 and rotational three degrees of freedom in the distal end portion 5, has been described as an example. However, the number of degrees of freedom of the robot R1 is not limited to six. The robot R1 may have less than six degrees of freedom or more than six degrees of freedom. In one embodiment, the robot R1 may have five or more degrees of freedom.

In the above embodiment, the case where the robot R2 has three degrees of freedom has been described as an example. However, the number of degrees of freedom of the robot R2 is not limited to three. As for the lower limit value, as described above, the robot R2 may have one or more degrees of freedom. Regarding the upper limit value, the robot R2 may have four or less degrees of freedom, for example. Four degrees of freedom include, for example, in addition to two degrees of freedom in the parallel link mechanism of the robot R2 and one degree of freedom in the insertion direction of the surgical tool T, one degree of freedom in rotation about the long axis of the surgical tool T such as forceps. By setting the number of degrees of freedom of the robot R2 to four or less, it is possible to prevent the degrees of freedom from becoming too redundant. In addition, in a case where the shape does not change even when the surgical tool T rotates in the long axis direction like an injection needle or the like, one degree of freedom in rotation is unnecessary, and the number of degrees of freedom of the robot R2 may be three or less.

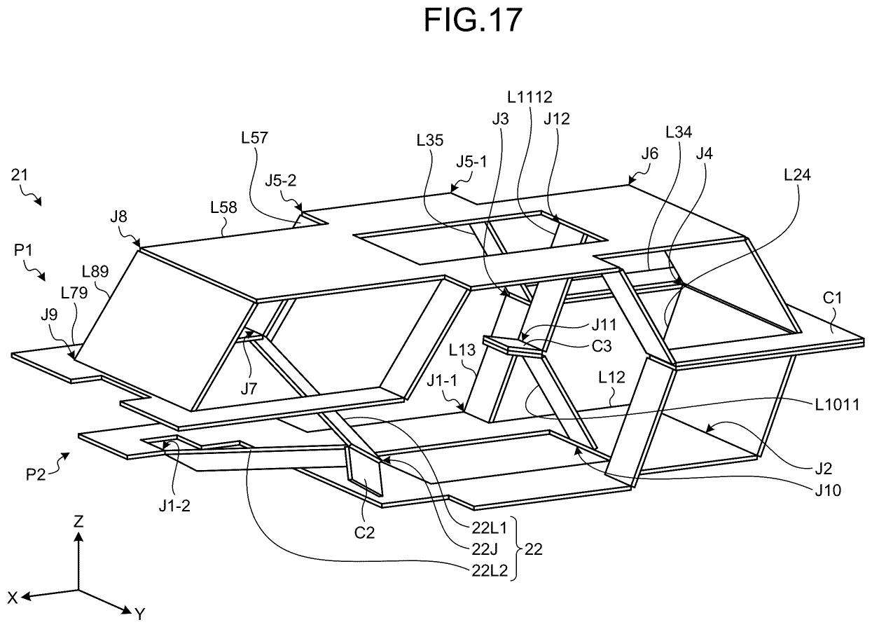

Various surgical tools may be used as the surgical tool T. Examples of the surgical tool T include forceps, an electric scalpel, an injection needle, an endoscope probe, and the like. For example, the surgical tool T as forceps is depicted in FIG. 1 described above. The surgical tool T as an injection needle will be described also with reference to FIG. 19.

FIG. 19 is a diagram illustrating an example of a schematic structure of a robot device 2. In this example, a surgical tool T supported by a robot R2 included in the robot device 2 is an injection needle. Since the injection needle only needs to move in the long axis direction, the robot R2 is only required to have only one degree of freedom in the insertion direction of the surgical tool T.

### 6. Embodiment of Retraction Mechanism

In one embodiment, a retraction mechanism may be provided between a robot R1 and a robot R2. This will be described with reference to FIG. 20 to FIG. 32.

FIG. 20 is a diagram illustrating an example of a schematic structure of a robot device 2. In this example, the robot device 2 further includes a retraction mechanism 101. The retraction mechanism 101 is provided between the robot R1 and the robot R2. For example, the retraction mechanism 101 is configured to retract the surgical tool T in a direction away from a surgical site in a case where the robot R2 supporting the surgical tool T causes a runaway or the like. The retraction may be active retraction performed by a user operation or may be passive retraction automatically performed without a user operation. Various known mechanisms configured to perform such retraction may be used as the retraction mechanism 101.

In addition, the retraction mechanism 101 may be configured to achieve both active retraction and passive retraction by combining nonlinear characteristics of the magnetic attraction force and linear characteristics of spring elasticity. Specific description will be given.

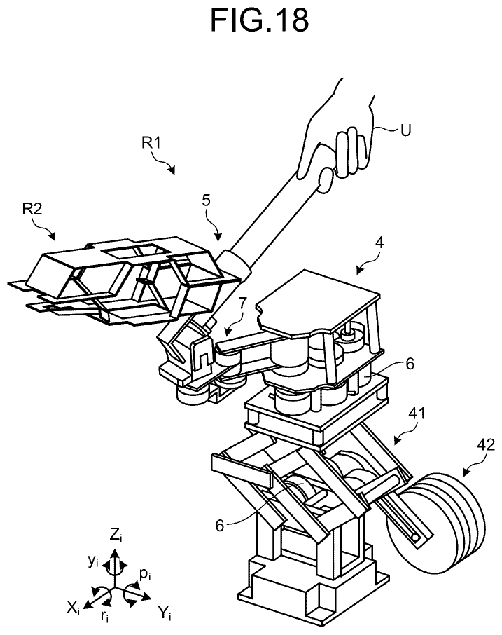

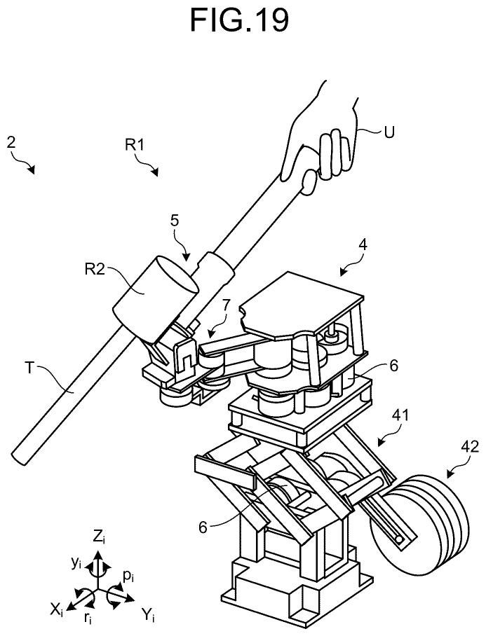

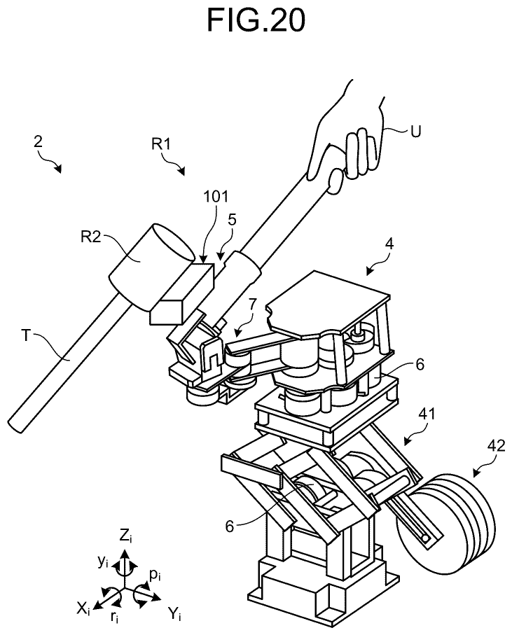

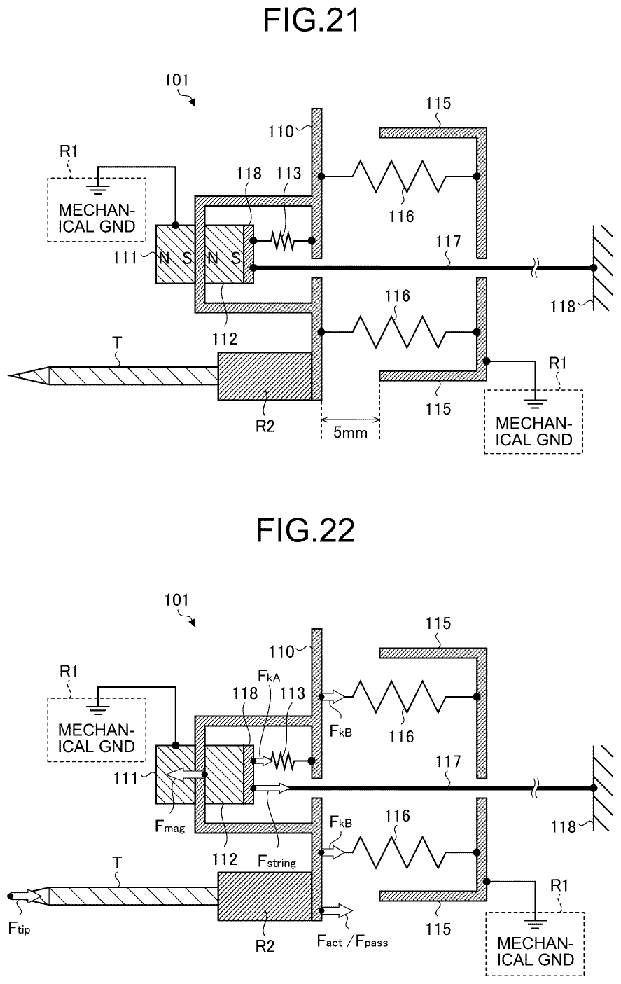

FIG. 21 is a schematic diagram illustrating a schematic structure example of the retraction mechanism 101. FIG. 22 is a diagram for explaining a force applied to each unit in the structure illustrated in FIG. 21. In FIG. 21, the robot R1 corresponds to mechanical GND. The retraction mechanism 101 includes a first member 118, a second member 110, a third member 115, magnets 111 and 112, springs 113 and 116, and a link mechanism 117. Note that, in the following description, in order to distinguish between the magnet 111 and the magnet 112, the magnet 111 is referred to as a fixed magnet 111, and the magnet 112 is referred to as an energizing magnet 112.

In the above structure, the fixed magnet 111 and the energizing magnet 112 constitute, for example, a first mechanism that energizes the first member 118 in a first direction (direction from the energizing magnet 112 towards the fixed magnet 111). Moreover, the second member 110 and the spring 113 constitute, for example, a second mechanism that energizes the first member 118 in a second direction opposite to the first direction. Furthermore, the springs 116 constitute a third mechanism that energizes the first member 118 in the second direction via the second member 110 and the energizing magnet 112.

The fixed magnet 111 and the third member 115 are fixed in the system of the retraction mechanism 101 (mechanical GND). For example, the fixed magnet 111 and the third member 115 are fixed to the distal end of the arm of the robot R1 on which the retraction mechanism 101 is mounted.

The second member 110 is disposed such that a position away from the third member 115 by a predetermined distance is a reference position. In other words, the second member 110 is separated from the third member 115 by a clearance distance in a state of being in contact with the fixed magnet 111.

The second member 110 is energized in a direction opposite to the fixed magnet 111 (rightward in the drawing) by the springs 116 having one end fixed to the third member 115. The springs 116 may be, for example, various springs such as a coil spring or a leaf spring. Furthermore, the springs 116 are not limited to a metal spring such as a coil spring or a leaf spring and may be, for example, a rubber spring, an air spring, a liquid spring, or the like. In addition, various elastic bodies such as a diaphragm may be used for the springs 116.

In addition, the robot R2 for supporting the surgical tool I for performing treatment on a patient is fixed to the second member 110. Note that, in the following description, the surgical tool T and the robot R2 may be collectively simply referred to as a surgical tool T. The direction in which the surgical tool T is attached to the second member 110 may be, for example, a direction (leftward in the drawing) opposite to the direction (rightward in the drawing) in which the second member 110 is energized by the springs 116.

The energizing magnet 112 is disposed in such a manner as to face the fixed magnet 111 with the second member 110 interposed therebetween. At this point, the energizing magnet 112 is disposed in such a manner that its magnetization direction (lateral direction in the drawing) exerts an attraction force with the fixed magnet 111.

The energizing magnet 112 is fixed to the first member 118. The first member 118 is energized by the spring 113 having one end fixed to the second member 110 in a direction opposite to the attracting direction between the fixed magnet 111 and the energizing magnet 112 (rightward in the drawing). Similarly to the springs 116, various elastic bodies such as a coil spring or a leaf spring may be used as the spring 113. Moreover, the elastic force of the spring 113 and the elastic forces of the springs 116 may have characteristics having different inclinations or may have characteristics having the same inclination. In the present embodiment, a case is described as an example in which, as the spring 113 and/or the springs 116, an elastic body having elastic characteristics in which the magnitude of the force linearly varies depending on the position of the first member 118 with respect to the fixed magnet 111 is used. However, it is not limited thereto, and an elastic body having elastic characteristics in which the magnitude of the force non-linearly varies may be used.

In the case of the configuration as the above, as illustrated in FIG. 22, the second member 110 is applied with a force (restoring force) k_{A}Δx_{A} (leftward force in the drawing) pushing towards the fixed magnet 111 by the energizing magnet 112 and a force (restoring force) k_{B}Δx_{B} (rightward force in the drawing) pulling towards the opposite side of the fixed magnet 111 by the springs 116. Note that, in the following description, the spring 113 is also referred to as a spring A, and the springs 116 are also referred to as springs B. Furthermore, k_{A} is a spring constant of the spring A, k_{B} is a spring constant of the springs B, Δx_{A} is a displacement amount of the spring A from the natural length, and Δx_{B} is a displacement amount of a spring B from the natural length.

Therefore, the force applied to the first member 118 by the fixed magnet 111, the energizing magnet 112, and the spring A supported by the second member 110 (denoted as a force f11) is obtained as a total value (f11=f_{mag}−k_{A}Δx_{A}) of the attraction force f_{mag} (leftward force in the drawing) between the fixed magnet 111 and the energizing magnet 112 and the force k_{A}Δx_{A} (rightward force in the drawing) by which the spring A pulls the first member 118.

Meanwhile, the force applied to the second member 110 by the fixed magnet 111, the energizing magnet 112, and the springs B supported by the third member 115 (which is referred to as a force f12) is obtained as a total value (f12=f_{mag}-k_{B}Δx_{B}) of the attraction force f_{mag} (leftward force in the drawing) between the fixed magnet 111 and the energizing magnet 112 and the force k_{B}Δx_{B} (rightward force in the drawing) by which the springs B pull the second member 110.

Let us return to the description of FIG. 21. The first member 118 to which the energizing magnet 112 is fixed is fixed with a second end of the link mechanism 117 having a first end attached to the first member 118 which is moved by the operation of an operator. The link mechanism 117 may be, for example, a string, a wire, a rod-shaped member, or the like. However, it is not limited thereto, and various modifications may be made as long as traction force can be remotely applied to the first member 118 (more specifically, the energizing magnet 112), such as the air pressure, the hydraulic pressure, a link, shape memory, a soft actuator, or the like.

In a case where a traction force fₛ (rightward force in the drawing) by the link mechanism 117 is within a range of the normal operation force by an operator, in other words, in a case where the traction force fₛ by the link mechanism 117 is less than or equal to the total force f11 (=f_{mag}−k_{A}Δx_{A}) of the attraction force f_{mag} between the fixed magnet 111 and the energizing magnet 112 and the restoring force k_{A}Δx_{A} by the spring A (fₛ≤f_{mag}−k_{A}Δx_{A}), the operator can operate the surgical tool T attached to the second member 110 by operating the link mechanism 117.

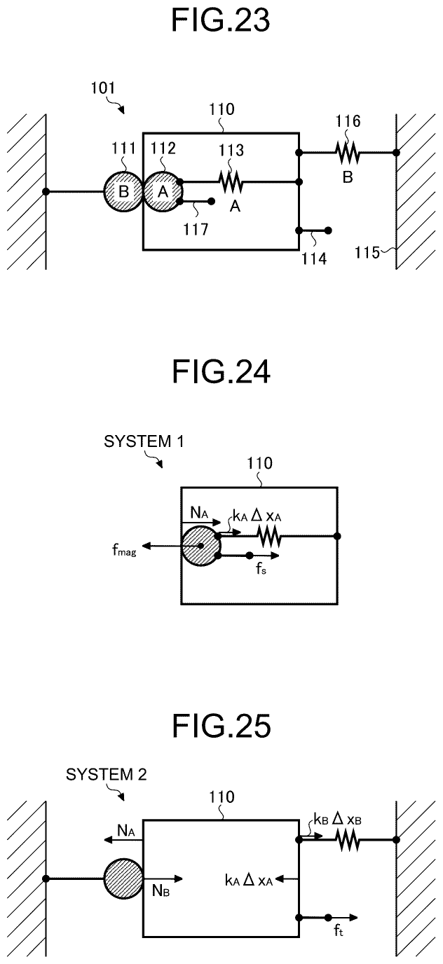

The operation principle of the retraction mechanism 101 will be described. Note that, for clarity of description, the structure of the retraction mechanism 101 according to the present embodiment is simplified herein as illustrated in FIG. 23.

In the simplified retraction mechanism 101 illustrated in FIG. 23 as an example, the first member 118 is omitted, and the spring 113 and the link mechanism 117 are directly attached to the energizing magnet 112. In the following description, the spring 113 is also referred to as a spring A, the spring 116 is also referred to as a spring B, the fixed magnet 111 is also referred to as a magnet B, and the energizing magnet 112 is also referred to as a magnet A.

Formed in such a structure are, as illustrated in FIG. 24, a system 1 that starts a retraction operation (active retraction operation) triggered by the traction force (internal force) fₛ of the link mechanism 117 reaching a first threshold value and, as illustrated in FIG. 25, a system 2 that starts a retraction operation (passive retraction operation) triggered by an external force fₜ applied to the surgical tool T reaching a second threshold value.

Note that, in the following description, mi may be the mass of the second member 110, m_{A} may be the mass of the energizing magnet 112 (magnet A), m_{B} may be the mass of the fixed magnet 111 (magnet B), N_{A} may be the normal force that the magnet A receives from the second member 110 in the system 1 and the normal force that the second member 110 receives from the magnet B in the system 2, N_{B} may be the normal force that the magnet A receives from the second member 110 in the system 2, f_{mag} may be the magnetic force (attraction force) between the magnets A and B, fₜ may be the acting force (external force) at the distal end of the surgical tool T, and fₛ may be the traction force (internal force) of the link mechanism 117.

With the above, in the system 1 illustrated in FIG. 24, the equation of motion of the magnet A (energizing magnet 112, which corresponds to the first member 118) can be expressed by the following equation (1), and in the system 2 illustrated in FIG. 25, the equation of motion of the second member 110 can be expressed by the following equation (2).

$$\{ \begin{matrix}System 1 : {m}_{A} {\ddot{x}}_{A} = {f}_{mag} - {k}_{A} Δ {x}_{A} - {f}_{s} - {N}_{A} ( 1 ) \\ System 2 : {m}_{1} {\ddot{x}}_{1} = - {k}_{B} Δ {x}_{B} - {f}_{t} - {N}_{B} + {N}_{A} + {K}_{A} Δ {x}_{A} ( 2 )\end{matrix}$$

From the above equations of motion (1) and (2), the stationary condition for both the system 1 and the system 2 to be stationary can be obtained as in the following equations (3) to (5).

$$\cdot From stationary condition : {\ddot{x}}_{A} = {\ddot{x}}_{1} = 0 \qquad ( 3 )$$

$$( 1 ) ⇔ {m}_{A} {\ddot{x}}_{A} = {f}_{mag} - ( {k}_{A} Δ {x}_{A} + {N}_{A} ) - {f}_{s}$$

$$( where {m}_{A} {\ddot{x}}_{A} = 0 )$$

$$⇔ {k}_{A} Δ {x}_{A} + {N}_{A} = {f}_{mag} - {f}_{s}$$

$$Substitute ( 3 ) into ( 2 ) \qquad ( 4 )$$

$$( 3 ) {⇔}^{( 2 )} {m}_{1} {\ddot{x}}_{1} = - {k}_{B} Δ {x}_{B} - {f}_{t} - {N}_{B} + {f}_{mag} - {f}_{s}$$

$$( where {m}_{1} {\ddot{x}}_{1} = 0 )$$

$$⇔ {N}_{B} = {f}_{mag} - {k}_{B} Δ {x}_{B} - ( {f}_{s} + {f}_{t} )$$

$$Transform ( 3 ) \qquad ( 5 )$$

$$( 3 ) ⇔ {N}_{A} = {f}_{mag} - {k}_{A} Δ {x}_{A} - {f}_{s}$$

Meanwhile, from the above equation of motion (1), the retraction condition for starting active retraction in the system 1 is expressed by the following equation (6), and from the above equation of motion (2), the retraction condition for starting passive retraction in the system 2 is expressed by the following equation (7).

$$Retraction condition of system 1$$

$$\cdot From retraction conditions : {N}_{A} < 0 , {N}_{B} < 0 \qquad ( 6 )$$

$$( 5 ) ⇔ {N}_{A} = {f}_{mag} - {k}_{A} Δ {x}_{A} - f < 0$$

$$⇔ {f}_{s} > {f}_{mag} - {k}_{A} Δ {x}_{A}$$

$$Retraction condition of system 2$$

$$( 4 ) ⇔ {N}_{B} = {f}_{mag} - {k}_{B} Δ {x}_{B} - ( {f}_{s} + {f}_{t} ) \qquad ( 7 )$$

$$⇔ {f}_{s} + {f}_{t} > {f}_{mag} - {k}_{B} Δ {x}_{B}$$

$$( 6 ) : {f}_{s} > {f}_{mag} - {k}_{A} Δ {x}_{A} ( {f}_{s} \geq 0 , Δ {x}_{A} \geq 0 )$$

$$( 7 ) : {f}_{s} + {f}_{t} > {f}_{mag} - {k}_{B} Δ {x}_{B} ( {f}_{t} \geq 0 , Δ {x}_{B} \geq 0 )$$

Here, by adjusting the initial position (displacement amount from the natural length at rest) of each of the spring A and the springs B in such a manner as to satisfy Δx_{A}>Δx_{B}, the force for satisfying the retraction condition of the system 1 (force serving as the trigger of active retraction=f_{mag}−k_{A}Δx_{A}) can be made smaller than the force for satisfying the retraction condition of the system 2 (force serving as the trigger of passive retraction=f_{mag}−k_{B}Δx_{B}) as in the following equation (8).

$${f}_{s} + {f}_{t} > {f}_{mag} - {k}_{B} Δ {x}_{B} > {f}_{s} > {f}_{mag} - {k}_{A} Δ {x}_{A} \qquad ( 8 )$$

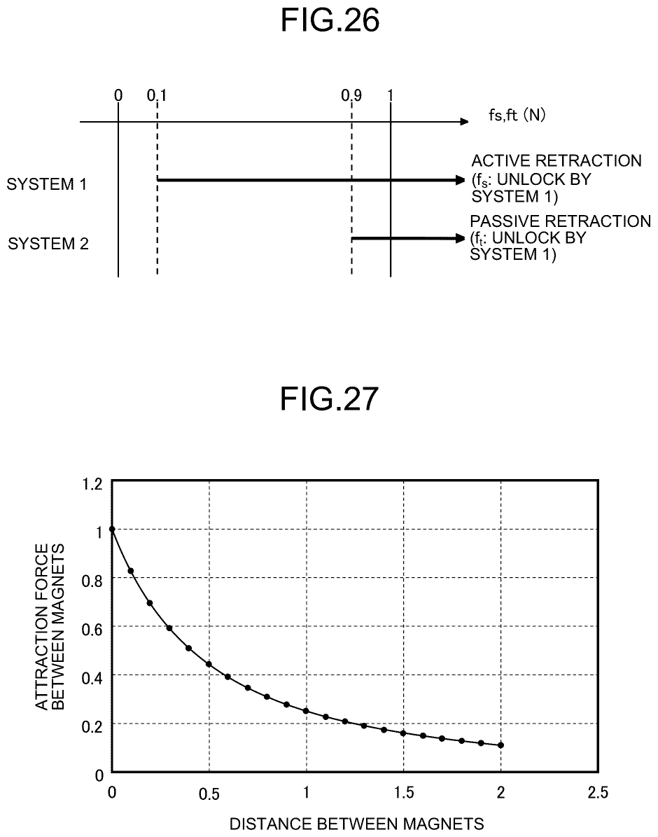

For example, in a case where it is designed such that the force (=f_{mag}−k_{A}Δx_{A}) as the trigger of the active retraction is 0.1 N and that the force (=f_{mag}−k_{B}Δx_{B}) as the trigger of the passive retraction is 0.9 N, as illustrated in FIG. 26, the active retraction is executed in a case where the traction force fₛ greater than or equal to 0.1 N is applied to the link mechanism 117, and the passive retraction is executed in a case where the external force fₜ greater than or equal to 0.9 N is applied to the surgical tool T.

As illustrated in FIG. 27 and the following equation (9), the attraction force f_{mag} between the fixed magnet 111 and the energizing magnet 112 has a characteristic of non-linearly varying depending on the position of the energizing magnet 112 or the first member 118 with respect to the fixed magnet 111. Specifically, the attraction force f_{mag} has a characteristic of becoming exponentially (non-linearly) small in inverse proportion to the distance between the magnets. Note that, in Equation (9), K is a constant, do is a distance between the magnets when the energizing magnet 112 comes closest to the fixed magnet 111 (corresponding to the thickness of the second member 110), and d is a distance that the energizing magnet 112 has moved in a direction away from the fixed magnet 111.

$$Attraction force between magnets : {f}_{mag} ( d ) = K \frac{1}{{( {d}_{0} + d )}^{2}} \qquad ( 9 )$$

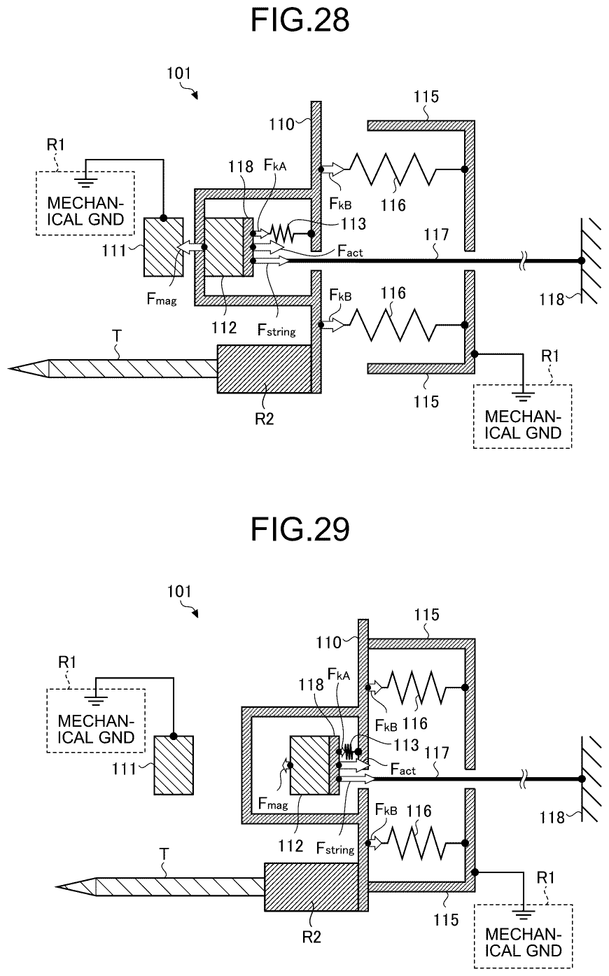

Next, an operation example in a case where the surgical tool T is automatically retracted (active retraction) in a case where an internal force (traction force fₛ) larger than a predetermined value is applied to the link mechanism 117 will be described. FIGS. 28 and 29 are diagrams for explaining an operation example at the time of active retraction according to the present embodiment.

When the traction force fₛ larger than the force f11 (=f_{mag}−k_{A}Δx_{A}) is applied to the link mechanism 117, from the above equation (6) of the retraction condition, the first member 118 starts to move in the direction away from the fixed magnet 111. At this point, since the second member 110 is pulled towards the third member 115 by the springs 116, the second member 110 moves towards the third member 115 as the first member 118 moves.

Subsequently, as illustrated in FIG. 29, when the energizing magnet 112 moves away from the fixed magnet 111 by a distance (d₀+d) at which the attraction force f_{mag} (d) between the magnets becomes smaller than the restoring force k_{A}Δx_{A} of the spring 113, the binding of the energizing magnet 112 by the fixed magnet 111 is released, and the first member 118 rapidly moves towards the third member 115 by a restoring force k_{A}Δx_{BA} of the spring 113 and the traction force fₛ of the link mechanism 117. Accordingly, since the support of the second member 110 by the energizing magnet 112 is released, the second member 110 rapidly moves towards the third member 115 by the restoring force k_{B}Δx_{B} of the springs 116 together with the surgical tool T (active retraction).

Then, the second member 110 moves by the clearance distance (for example, 5 mm) between the second member 110 and the third member 115 and comes into contact with the third member 115 to stop.

As described above, in the retraction mechanism 101 according to the present embodiment, in a case where the traction force fₛ larger than the force (f11=f_{mag}−k_{A}Δx_{A}) designed in advance is applied to the link mechanism 117, that is, in a case where the operator who operates the surgical tool T intentionally or negligently causes the traction force fₛ larger than the force (f11=f_{mag}−k_{A}Δx_{A}) designed in advance to be generated in the link mechanism 117, it is possible to release the lock by the fixed magnet 111 and to automatically retract the surgical tool T in a safe direction.

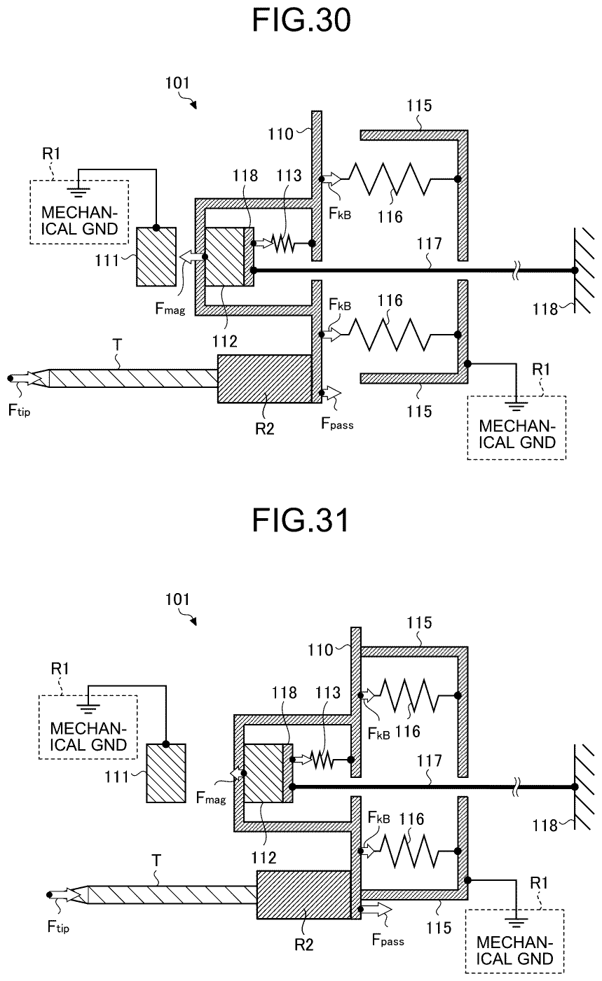

Next, an operation example in a case where the surgical tool T is automatically retracted in a case where the external force fₜ larger than a set value is applied to the surgical tool T (passive retraction) will be described. FIGS. 30 and 31 are diagrams for explaining an operation example at the time of passive retraction according to the present embodiment.

When a force fₛ+fₜ larger than the force f12 (=f_{mag}−k_{B}Δx_{B}) is applied to the surgical tool T and the link mechanism 117 from the above equation (7) of the retraction condition, the second member 110 starts to move in the direction away from the fixed magnet 111. At this point, since the energizing magnet 112 is in contact with the second member 110 due to the first member 118 energized by the spring 113, the energizing magnet 112 moves towards the third member 115 as the second member 110 moves.

Subsequently, as illustrated in FIG. 31, when the energizing magnet 112 moves away from the fixed magnet 111 by a distance (d₀+d) at which the attraction force f_{mag} (d) between the magnets is smaller than the restoring force k_{B}Δx_{B} of the springs 116, the binding of the energizing magnet 112 by the fixed magnet 111 is released, and the second member 110 rapidly moves towards the third member 115 by the restoring force k_{B}Δx_{B} of the spring 116 and the force fₛ+fₜ applied to the surgical tool T and the link mechanism 117 (passive retraction).

Then, the second member 110 moves by the clearance distance (for example, 5 mm) between the second member 110 and the third member 115 and comes into contact with the third member 115 to stop.

As described above, in the retraction mechanism 101 according to the present embodiment, in a case where the force fₛ+fₜ larger than the force (f12=f_{mag}−k_{B}Δx_{B}) designed in advance is applied to the surgical tool T and the link mechanism 117, it is possible to release the lock by the fixed magnet 111 and to automatically retract the surgical tool T in a safe direction.

In the above retraction mechanism 101, the system 1 that implements active retraction and the system 2 that implements passive retraction are configured by different systems. Therefore, the systems can be automatically executed using forces having different magnitudes as triggers. For example, it is possible to implement both the active retraction by an operation force that is ergonomically appropriate as well as the passive retraction by an operation force corresponding to an external force.

### 7. Exemplary Effects

The disclosed technology may be specified as follows. The robot R1 may have five or more degrees of freedom. The robot R2 may have four or less degrees of freedom. Moreover, the robot R2 may include an actuator and be actively driven. For example, by using such a robot R1 and robot R2, it is made possible to prevent the degrees of freedom from becoming redundant and excessive while ensuring the necessary operation.

As described with reference to FIGS. 20 to 32 and others, the robot device 2 may include the safety retraction mechanism 101 and others provided between the robot R1 and the robot R2, and the retraction mechanism may include the first member 118, the second member 110, the first mechanism that stops the first member 118 by energizing the first member 118 towards the second member 110, the second mechanism that releases the stop of the first member 118 by the first mechanism depending on the force applied to the first member 118, and the third mechanism that releases the stop of the first member 118 by the first mechanism depending on the force applied to the second member 110. This makes it possible to safely perform the retraction operation.

As described with reference to FIG. 20 and others, the surgical tool T may include an injection needle. Aligning of such a surgical tool T is also possible.

Although the embodiments of the disclosure have been described above, the technical scope of the disclosure is not limited to the above embodiments as they are, and various modifications can be made without departing from the gist of the disclosure. In addition, components of different embodiments and modifications may be combined as appropriate.

Note that the present technology can also have the following structures.

(1) A robot device comprising:
a first robot including a base portion and a distal end portion; and
a second robot supported by the distal end portion of the first robot, the second robot supporting a surgical tool to be inserted into a body of a patient,
wherein the first robot is operated by a user by directly applying a force to the first robot.

(2) The robot device according to (1),
wherein the first robot includes a lock mechanism that locks a joint.

(3) The robot device according to (2),
wherein the lock mechanism includes an electromagnetic brake that unlocks the joint when applied with a voltage and locks the joint when applied with no voltage.

(4) The robot device according to (2) or (3),
wherein the lock mechanism is provided to the base portion.

(5) The robot device according to any one of (2) to (4),
wherein the first robot includes a transmission that transmits a braking force from the lock mechanism to the joint, and
the transmission transmits the braking force from the lock mechanism to the joint using at least one of a wire, a wire rope, a belt, a steel belt, a hydraulic pressure, a pneumatic pressure, a dielectric elastomer, or a shape memory alloy.

(6) The robot device according to (5),
wherein the transmission is provided to the base portion.

(7) The robot device according to any one of (1) to (6),
wherein the base portion has translational three degrees of freedom, and
the distal end portion has rotational two or more degrees of freedom.

(8) The robot device according to any one of (1) to (7),
wherein the second robot is detachably attached to the distal end portion of the first robot.

(9) The robot device according to any one of (1) to (8),
wherein the second robot can be remotely controlled.

(10) The robot device according to any one of (1) to (9),
wherein the surgical tool has a marker physically provided at a position corresponding to a remote center of motion of the surgical tool.

(11) The robot device according to any one of (1) to (10),
wherein the first robot has a size that allows a user to grip and operate the first robot with one hand, and
the second robot is smaller than the first robot.

(12) The robot device according to any one of (1) to (11),
wherein the surgical tool is inserted into an eyeball of the patient.

(13) The robot device according to any one of (1) to (12),
wherein the first robot has five or more degrees of freedom,
the second robot has four or less degrees of freedom, and
the second robot includes an actuator and is actively driven.

(14) The robot device according to any one of (1) to (13), further comprising:
a retraction mechanism provided between the first robot and the second robot,
wherein the retraction mechanism includes:
a first member;
a second member;
a first mechanism that stops the first member by energizing the first member towards the second member;
a second mechanism that releases a stop of the first member by the first mechanism depending on a force applied to the first member; and
a third mechanism that releases the stop of the first member by the first mechanism depending on a force applied to the second member.

(15) The robot device according to any one of (1) to (14),
wherein the surgical tool includes an injection needle.

(16) A robot system comprising:
a robot device; and
a support device,
wherein the robot device includes:
a first robot including a base portion and a distal end portion; and
a second robot supported by the distal end portion of the first robot, the second robot supporting a surgical tool to be inserted into a body of a patient,
the first robot is operated by a user by directly applying a force to the first robot, and
the support device supports operation of the first robot by the user for aligning an insertion point of the surgical tool and a remote center of motion of the surgical tool.

(17) The robot system according to (16),
wherein the support device notifies the user of a positional deviation between the insertion point of the surgical tool and the remote center of motion of the surgical tool.

(18) The robot system according to (16),
wherein the support device detects a positional deviation between the insertion point of the surgical tool and the remote center of motion of the surgical tool on a basis of a change in observation images of a microscope that observes a surgical field when the surgical tool rotates.

(19) The robot system according to (16),
wherein the support device detects a positional deviation between the insertion point of the surgical tool and the remote center of motion of the surgical tool on a basis of a reaction force from the insertion point of the surgical tool at a time of rotation of the surgical tool.

(20) The robot system according to (16),
wherein the support device notifies the user U that the insertion point of the surgical tool and a position of a marker physically provided at a position corresponding to the remote center of motion in the surgical tool coincide with each other.

(21) The robot device according to any one of (1) to (20), further including:
a parallel link mechanism extending in a plane direction of a first plane; and
a connection mechanism connected to the parallel link mechanism,
in which the parallel link mechanism includes:
a support link that supports, on a distal end side, a surgical tool to be inserted into a body of a patient;
an opposing link facing the support link;
a first joint connected to a first end of the opposing link;
a second joint connected to a second end of the opposing link; and
a third joint disposed at a base, the base being an end on a side opposite to the distal end side, the third joint rotationally driven, and
the connection mechanism is connected between the second joint and the third joint, the connection mechanism transformed in a plane direction of a second plane intersecting the first plane in such a manner that the second joint moves relative to the third joint in an extending direction of the opposing link.

(22) The robot device according to (21), further including:
a bendable plate-shaped member that forms the parallel link mechanism and the connection mechanism,
in which a bent portion of the plate-shaped member functions as a joint, and
a portion connecting bent portions in the plate-shaped member functions as a link.

(23) The robot device according to (22),
in which the bent portion of the plate-shaped member is elastically transformable, and
the portion connecting the bent portions of the plate-shaped member has higher rigidity than rigidity of the bent portions.

(24) The robot device according to (22),
in which the plate-shaped member is a plurality of plate-shaped members partially bonded to each other.

### REFERENCE SIGNS LIST

1 ROBOT SYSTEM
2 ROBOT DEVICE
3 MONITOR
4 BASE PORTION
41 TRANSLATION MECHANISM
42 COUNTERWEIGHT
5 DISTAL END PORTION
51 ROTATION MECHANISM
6 LOCK MECHANISM
7 TRANSMISSION
8 SUPPORT DEVICE
MC MICROSCOPE
E EYEBALL
F FIELD OF VIEW
I INSERTION POINT
M MARKER
O OBJECT
RCM REMOTE CENTER OF MOTION (FIXED POINT IN PIVOT ROTATION)
R1 ROBOT
R2 ROBOT
R3 ROBOT
T SURGICAL TOOL
U USER
20 SUPPORT ARM DEVICE
21 PARALLEL LINK MECHANISM
22 CONNECTION MECHANISM
22J JOINT
22L1 LINK
22L2 LINK
22A CONNECTION MECHANISM
22AJ1 JOINT
22AL1 LINK
22AL2 LINK
22AL3 LINK
22AJ3 JOINT
22AL4 LINK
22B CONNECTION MECHANISM
22C CONNECTION MECHANISM
23 SUPPORT MEMBER
C1 BONDING PORTION
C2 BONDING PORTION
C3 BONDING PORTION
J1 JOINT
J2 JOINT
J3 JOINT
J4 JOINT
J5 JOINT
J6 JOINT
J7 JOINT
J8 JOINT
J9 JOINT
J10 JOINT
J11 JOINT
J12 JOINT
L12 LINK
L13 LINK
L24 LINK
L34 LINK
L35 LINK
L46 LINK
L57 LINK
L58 LINK
L79 LINK
L89 LINK
L1011 LINK
L1112 LINK
P1 PLATE-SHAPED MEMBER
P2 PLATE-SHAPED MEMBER
101 RETRACTION MECHANISM
110 SECOND MEMBER
111 MAGNET (FIXED MAGNET)
112 MAGNET (ENERGIZING MAGNET)
113 SPRING
115 THIRD MEMBER
116 SPRING
117 LINK MECHANISM
118 FIRST MEMBER

1. A robot device comprising:
a first robot including a base portion and a distal end portion; and
a second robot supported by the distal end portion of the first robot, the second robot supporting a surgical tool to be inserted into a body of a patient,
wherein the first robot is operated by a user by directly applying a force to the first robot.

2. The robot device according to claim 1,
wherein the first robot includes a lock mechanism that locks a joint.

3. The robot device according to claim 2,
wherein the lock mechanism includes an electromagnetic brake that unlocks the joint when applied with a voltage and locks the joint when applied with no voltage.

4. The robot device according to claim 2,
wherein the lock mechanism is provided to the base portion.

5. The robot device according to claim 2,
wherein the first robot includes a transmission that transmits a braking force from the lock mechanism to the joint, and
the transmission transmits the braking force from the lock mechanism to the joint using at least one of a wire, a wire rope, a belt, a steel belt, a hydraulic pressure, a pneumatic pressure, a dielectric elastomer, or a shape memory alloy.

6. The robot device according to claim 5,
wherein the transmission is provided to the base portion.

7. The robot device according to claim 1,
wherein the base portion has translational three degrees of freedom, and
the distal end portion has rotational two or more degrees of freedom.

8. The robot device according to claim 1,
wherein the second robot is detachably attached to the distal end portion of the first robot.

9. The robot device according to claim 1,
wherein the second robot can be remotely controlled.

10. The robot device according to claim 1,
wherein the surgical tool has a marker physically provided at a position corresponding to a remote center of motion of the surgical tool.

11. The robot device according to claim 1,
wherein the first robot has a size that allows a user to grip and operate the first robot with one hand, and
the second robot is smaller than the first robot.

12. The robot device according to claim 1,
wherein the surgical tool is inserted into an eyeball of the patient.

13. The robot device according to claim 1,
wherein the first robot has five or more degrees of freedom,
the second robot has four or less degrees of freedom, and
the second robot includes an actuator and is actively driven.

14. The robot device according to claim 1, further comprising:
a retraction mechanism provided between the first robot and the second robot,
wherein the retraction mechanism includes:
a first member;
a second member;
a first mechanism that stops the first member by energizing the first member towards the second member;
a second mechanism that releases a stop of the first member by the first mechanism depending on a force applied to the first member; and
a third mechanism that releases the stop of the first member by the first mechanism depending on a force applied to the second member.

15. The robot device according to claim 1,
wherein the surgical tool includes an injection needle.

16. A robot system comprising:
a robot device; and
a support device,
wherein the robot device includes:
a first robot including a base portion and a distal end portion; and
a second robot supported by the distal end portion of the first robot, the second robot supporting a surgical tool to be inserted into a body of a patient,
the first robot is operated by a user by directly applying a force to the first robot, and
the support device supports operation of the first robot by the user for aligning an insertion point of the surgical tool and a remote center of motion of the surgical tool.

17. The robot system according to claim 16,
wherein the support device notifies the user of a positional deviation between the insertion point of the surgical tool and the remote center of motion of the surgical tool.

18. The robot system according to claim 16,
wherein the support device detects a positional deviation between the insertion point of the surgical tool and the remote center of motion of the surgical tool on a basis of a change in observation images of a microscope that observes a surgical field when the surgical tool rotates.

19. The robot system according to claim 16,
wherein the support device detects a positional deviation between the insertion point of the surgical tool and the remote center of motion of the surgical tool on a basis of a reaction force from the insertion point of the surgical tool at a time of rotation of the surgical tool.

20. The robot system according to claim 16,
wherein the support device notifies the user U that the insertion point of the surgical tool and a position of a marker physically provided at a position corresponding to the remote center of motion in the surgical tool coincide with each other.
